(19) United States
(12) Patent Application Publication
(10) Pub. No.: US 2026/0335615 A1
(43) Pub. Date: Oct. 29, 2026

(54) INTERNAL ANAL SPHINCTER LOCALIZATION AND INJECTION DEVICE

(71) Applicants: Maha Ahmed Alamodi ALGHAMDI, Abha (SA); Abdullah Ahmed ALAMOUDI, Abha (SA); Hemali DESHPANDE, Abha (SA)

(72) Inventors: Maha Ahmed Alamodi ALGHAMDI, Abha (SA); Abdullah Ahmed ALAMOUDI, Abha (SA); Hemali DESHPANDE, Abha (SA)

(21) Appl. No.: 19/701,588

(22) Filed: Jun. 8, 2026

(51) Int. Cl. A61M 31/00; A61B 5/06; A61B 5/389

(52) U.S. Cl. CPC A61M 31/00; A61B 5/063; A61B 5/389

(57) ABSTRACT

The present invention discloses internal anal sphincter localization and injection system configured for targeted delivery of therapeutic agents into internal anal sphincter tissue through a machine-assisted localization and controlled deployment arrangement. The system comprises an elongated probe structure having a proximal handling portion and a distal insertion portion configured for placement within an anal canal. A tissue sensing unit positioned on the distal insertion portion includes multiple sensing structures configured to acquire tissue response information from surrounding anatomical regions. A signal acquisition processor receives sensing information and performs signal conditioning and data acquisition operations, while a tissue differentiation processor generates tissue characterization information corresponding to tissue boundaries and muscular structures. A deployment authorization processor evaluates tissue identification information and permits therapeutic administration only after confirmation of internal anal sphincter localization.

### TECHNICAL FIELD

The present invention generally relates to therapeutic localization and minimally invasive intervention systems and more specifically relates to a structurally integrated machine-assisted internal anal sphincter localization and injection device configured to identify tissue structures within an anorectal region and deliver therapeutic agents through a controlled deployment mechanism.

### BACKGROUND OF THE INVENTION

Chronic anal fissures frequently arise from sustained hypertonicity and spasm of the internal anal sphincter. Therapeutic approaches involving administration of pharmacological agents including neurotoxic muscular relaxants have demonstrated effectiveness in reducing sphincter pressure and promoting tissue healing. Existing delivery procedures generally involve manual injections based upon anatomical estimates or clinician experience. Such procedures frequently suffer from uncertainty regarding tissue localization because the internal anal sphincter is not externally visible and exhibits anatomical variations among individuals.

Conventional injection methods may therefore lead to unintended deposition of therapeutic material into adjacent structures including the external anal sphincter or surrounding mucosal layers. Such inaccuracies can produce reduced treatment efficacy and potential complications including muscular dysfunction and variable therapeutic response. Procedural success therefore becomes heavily dependent on operator expertise and subjective judgment.

Accordingly, a need exists for a structurally integrated localization and injection machine capable of identifying tissue interfaces and permitting therapeutic delivery only after positive identification of the desired muscular layer.

Chronic anorectal disorders, particularly chronic anal fissures, represent a significant therapeutic challenge due to the complex anatomical and physiological characteristics of the anal canal and sphincter structures. Chronic anal fissures are generally associated with elevated resting pressure and persistent hypertonicity of the internal anal sphincter, resulting in reduced local blood perfusion, impaired tissue healing, and recurring pain. Clinical management frequently involves reduction of sphincter spasm through pharmacological or procedural interventions intended to decrease resting muscular tone and restore normal tissue perfusion. Among non-surgical treatment modalities, localized injection of therapeutic agents such as botulinum toxin into the internal anal sphincter has gained widespread clinical acceptance due to its ability to induce temporary muscular relaxation without requiring invasive surgical procedures. Therapeutic efficacy of such treatment, however, depends substantially upon accurate placement of the injected agent directly into the internal anal sphincter tissue. Improper localization may significantly reduce therapeutic effectiveness and potentially introduce undesirable complications.

The anatomy of the anorectal region presents substantial technical challenges for targeted therapeutic delivery procedures. The internal anal sphincter is composed predominantly of involuntary smooth muscle and is located beneath mucosal tissues while remaining adjacent to the external anal sphincter, which comprises voluntary skeletal muscle structures. Due to close spatial proximity and overlapping tissue boundaries, differentiation between these tissue structures during therapeutic procedures becomes difficult. Internal tissue layers are not externally visible and often exhibit patient-specific anatomical variations associated with age, muscular development, tissue pathology, and prior surgical interventions. Such anatomical complexity frequently limits procedural precision and increases dependence upon practitioner experience.

A further limitation associated with existing therapeutic delivery solutions involves absence of integrated safety mechanisms configured to prevent injection unless target tissue identification has been confirmed. Most existing systems allow deployment regardless of localization certainty. In situations involving inaccurate positioning, therapeutic administration may proceed despite uncertainty regarding target tissue identification. Lack of procedural interlocks introduces risk associated with accidental injection into unintended anatomical structures.

Therefore, despite numerous developments in therapeutic localization and delivery technologies, existing solutions continue to exhibit limitations involving procedural variability, operator dependence, inadequate tissue discrimination, limited integration of sensing and delivery architectures, insufficient anatomical adaptability, and absence of integrated safety-controlled deployment mechanisms. Consequently, a need remains for an integrated machine-assisted localization and injection structure capable of combining tissue sensing, tissue differentiation, controlled penetration, and safety-regulated therapeutic delivery within a single dedicated medical device platform configured to improve localization accuracy, reduce procedural uncertainty, and enhance therapeutic outcomes.

### SUMMARY OF THE INVENTION

The present invention provides an internal anal sphincter localization and injection device comprising a structural insertion probe assembly, an integrated tissue characterization subsystem, a deployable therapeutic delivery structure, and a controlled activation architecture configured to permit tissue penetration only after confirmation of target tissue identification.

In one embodiment, the device comprises an elongated insertion structure extending between a proximal handling section and a distal localization section. The distal section includes a circumferential tissue sensing array configured to detect multiple tissue characteristics and generate tissue differentiation information during insertion and positioning operations.

The sensing arrangement may comprise ultrasound sensing elements, electrophysiological sensing elements, impedance measurement structures, tissue-contact sensing pads, pressure-responsive interfaces, or combinations thereof. Data obtained from the sensing arrangement may be processed by an integrated hardware processing architecture configured to identify tissue transitions between mucosal layers, internal sphincter tissue, and external sphincter tissue.

Upon confirmation of target tissue localization, a deployment assembly permits extension of a therapeutic delivery needle through a controlled penetration pathway toward the identified region. The penetration depth is mechanically restricted by a depth-limiting structure to reduce unintended penetration into neighboring tissue layers.

The invention thereby transforms a conventionally operator-dependent procedure into a guided localization and controlled injection process through an integrated machine architecture.

The primary object of the present invention is to provide an internal anal sphincter localization and injection device configured to accurately identify internal sphincter tissue and facilitate controlled delivery of a therapeutic agent into the identified tissue region with improved procedural precision. The invention seeks to establish a structurally integrated medical device architecture capable of combining tissue localization and therapeutic administration functions within a single machine-assisted platform. The object is derived from the disclosed localization and injection system structure and therapeutic objectives described in the source disclosure.

Another object of the present invention is to provide a therapeutic localization and administration system capable of reducing treatment variability, minimizing complications associated with inaccurate injection placement, improving consistency of therapeutic outcomes, and enhancing patient safety during treatment of chronic anorectal conditions.

### BRIEF DESCRIPTION OF FIGURES

These and other features, aspects, and advantages of the present invention will become better understood when the following detailed description is read concerning the accompanying drawings in which like characters represent like parts throughout the drawings, wherein:

FIG. 1 displays a block diagram of an internal anal sphincter localization and injection system for targeted delivery of a therapeutic agent into an internal anal sphincter region;

FIG. 2 illustrates a perspective view of a tissue-targeted therapeutic delivery device (100) configured to perform localized tissue sensing, target differentiation, controlled penetration, and therapeutic delivery within a selected anatomical region;

FIG. 3 illustrates a cross-sectional view of a distal probe portion positioned within a target tissue region; and

FIG. 4 illustrates a flow chart of a method for targeted delivery of a therapeutic agent into an internal anal sphincter region.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises...a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

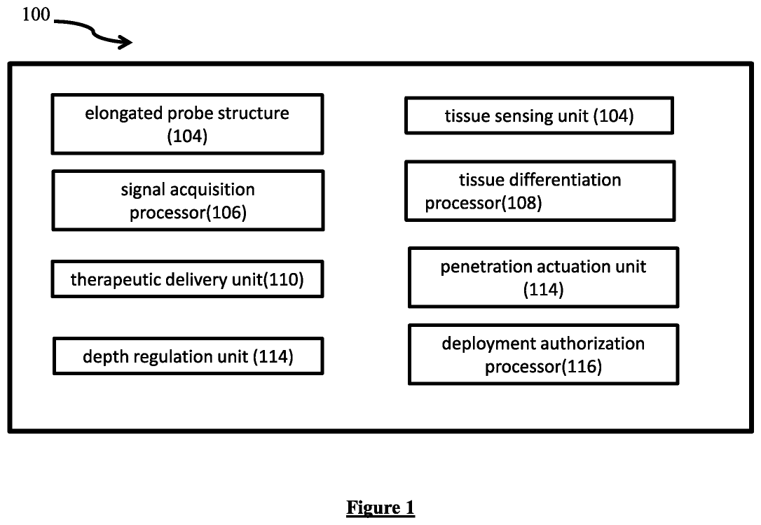

Referring to FIG. 1, a block diagram of an internal anal sphincter localization and injection system for targeted delivery of a therapeutic agent into an internal anal sphincter region is illustrated. The system 100 comprises: an elongated probe structure (102) comprising a proximal handling portion and a distal insertion portion configured for placement within an anal canal; a tissue sensing unit (104) disposed at the distal insertion portion and comprising a plurality of tissue interaction elements positioned circumferentially around an outer surface of the elongated probe structure; a signal acquisition processor (106) electrically coupled to the tissue sensing unit and configured to receive tissue response information generated by the plurality of tissue interaction elements; a tissue differentiation processor (108) operatively coupled to the signal acquisition processor and configured to generate tissue characterization information corresponding to anatomical tissue layers surrounding the distal insertion portion; a therapeutic delivery unit (110) positioned within an internal cavity of the elongated probe structure and comprising a retractable penetration structure configured to selectively extend from a deployment opening formed in the distal insertion portion; a penetration actuation unit (112) mechanically coupled to the therapeutic delivery unit and configured to produce controlled displacement of the retractable penetration structure; a depth regulation unit (114) coupled to the penetration actuation unit and configured to restrict advancement distance of the retractable penetration structure; and a deployment authorization processor (116) operatively coupled to the tissue differentiation processor and the penetration actuation unit, wherein the deployment authorization processor is configured to permit extension of the retractable penetration structure only after identification of tissue characteristics corresponding to an internal anal sphincter region.

In an embodiment, the the tissue sensing unit comprises a circumferential acoustic sensing arrangement including a plurality of high-frequency ultrasound transducer elements distributed at angular intervals around the distal insertion portion, each high-frequency ultrasound transducer element being configured to emit acoustic signals toward surrounding tissue structures and receive reflected signals representative of tissue interface boundaries, and wherein the signal acquisition processor is configured to generate spatial tissue profile information from time-varying acoustic reflections obtained from the plurality of high-frequency ultrasound transducer elements, and wherein the tissue sensing unit further comprises an electrophysiological sensing arrangement including multiple annular conductive sensing elements embedded beneath an insulating surface layer disposed around the elongated probe structure, the conductive sensing elements being configured to establish temporary contact with adjacent tissue and detect electrical activity associated with muscular tissue structures, and wherein the tissue differentiation processor is configured to distinguish smooth muscle tissue characteristics from skeletal muscle tissue characteristics using electrical activity variations acquired through the conductive sensing elements.

In an embodiment, the electrophysiological sensing arrangement further comprises circumferentially segmented electrode regions positioned in axially separated rows along the distal insertion portion, each segmented electrode region being individually addressable by the signal acquisition processor to permit sequential acquisition of localized tissue activity information and generation of three-dimensional muscular activity distribution data corresponding to surrounding anatomical structures, and wherein the tissue sensing unit comprises an impedance sensing arrangement including multiple conductive contact regions configured to introduce electrical excitation signals into adjacent tissue structures and measure electrical resistance characteristics associated with tissue composition, and wherein the tissue differentiation processor is configured to determine transitions among mucosal tissue, internal sphincter tissue, and external sphincter tissue based upon measured electrical response characteristics and spatial impedance distribution information.

The above embodiment may be implemented through an integrated multimodal sensing architecture in which the distal insertion portion of the elongated probe structure carries a plurality of circumferentially segmented electrode regions arranged in multiple axially separated sensing rows. The electrode regions may be fabricated from highly conductive and biocompatible materials including platinum-iridium alloys, gold-coated conductive substrates, titanium nitride coated conductors, stainless steel sensing contacts, conductive ceramic elements, or combinations thereof. The circumferential segmentation permits individual sectors of tissue surrounding the probe to be interrogated independently, while the axial separation of sensing rows enables acquisition of tissue activity information at different longitudinal positions along the anal canal. Each electrode segment is electrically coupled to a multiplexed signal acquisition processor through shielded conductive pathways and switching circuitry configured to selectively activate individual electrode regions according to predefined sensing sequences. As the probe is positioned within the anal canal, the processor sequentially acquires localized electrophysiological information from multiple circumferential and longitudinal locations, thereby creating a spatially resolved dataset representative of muscular activity occurring within surrounding anatomical structures.

The acquired electrophysiological information may include resting electrical potentials, spontaneous smooth muscle activity, neuromuscular conduction characteristics, electrical signal amplitudes, frequency distributions, phase relationships, propagation patterns, and temporal activation characteristics associated with adjacent tissues. Since the internal anal sphincter primarily comprises involuntary smooth muscle tissue while the external anal sphincter comprises voluntary skeletal muscle tissue, the electrical activity patterns generated by these structures differ significantly. The processor therefore analyzes variations in signal amplitude, activation periodicity, conduction velocity, signal synchronization, and circumferential distribution to distinguish between the different muscular regions. By combining measurements obtained from multiple electrode segments positioned at different circumferential and longitudinal locations, the processor reconstructs a three-dimensional muscular activity distribution map representing the physiological behavior of tissues surrounding the distal insertion portion. Such mapping enables visualization of muscular continuity, circumferential symmetry, localized muscular defects, and transition regions between adjacent anatomical structures.

In addition to electrophysiological sensing, the tissue sensing unit incorporates an impedance sensing arrangement configured to evaluate electrical properties of tissue located adjacent to the probe. The impedance sensing arrangement may include multiple conductive contact regions distributed around the distal insertion portion and electrically coupled to controlled excitation circuitry. During operation, the excitation circuitry generates low-amplitude alternating current signals at one or more selected frequencies and introduces such signals into surrounding tissue through selected conductive contact regions. Corresponding voltage responses are detected by neighboring contact regions, enabling determination of tissue impedance characteristics. The excitation signals may be generated at multiple frequencies ranging from a few kilohertz to several megahertz to obtain frequency-dependent impedance information associated with cellular architecture, extracellular fluid concentration, membrane capacitance, tissue density, and muscular composition.

Different tissue layers surrounding the anal canal exhibit distinct electrical impedance characteristics due to differences in cellular organization, hydration levels, collagen content, vascularization, muscle fiber arrangement, and extracellular matrix composition. For example, mucosal tissue generally exhibits impedance characteristics associated with epithelial and submucosal structures, whereas smooth muscle tissue forming the internal anal sphincter exhibits a different impedance profile resulting from densely organized involuntary muscle fibers. Similarly, skeletal muscle tissue of the external anal sphincter demonstrates impedance responses influenced by fiber orientation, contractile properties, and neuromuscular organization. The impedance sensing arrangement therefore generates a spatial impedance distribution profile indicative of tissue composition surrounding the probe.

The tissue differentiation processor receives the electrophysiological information and impedance information and performs multimodal tissue characterization using correlation algorithms, tissue classification models, pattern recognition procedures, and spatial mapping techniques. The processor may first construct circumferential impedance maps corresponding to individual sensing levels and thereafter correlate the impedance information with localized muscular activity information generated from the segmented electrode regions. Regions exhibiting impedance characteristics associated with smooth muscle tissue together with electrophysiological signatures corresponding to involuntary muscular activity are identified as candidate internal anal sphincter regions. Conversely, regions exhibiting impedance responses characteristic of skeletal muscle tissue combined with voluntary muscular activation signatures are classified as external sphincter tissue. Areas exhibiting electrical properties associated with epithelial and connective tissue structures together with minimal muscular activity may be identified as mucosal tissue.

In an exemplary procedure, the probe may initially be inserted into the anal canal while sequential sensing cycles are continuously performed. During advancement, a first circumferential sensing row may detect relatively low muscular activity together with impedance values characteristic of mucosal tissue. As the distal insertion portion approaches the internal sphincter region, the processor may detect a circumferentially continuous increase in smooth-muscle-associated electrical activity accompanied by impedance characteristics corresponding to dense involuntary muscular tissue. Upon further advancement, a subsequent sensing row may detect impedance and electrophysiological characteristics indicative of skeletal muscle activity associated with the external sphincter. By analyzing these spatial transitions across multiple sensing rows and circumferential sectors, the processor determines the precise boundaries separating mucosal tissue, internal sphincter tissue, and external sphincter tissue.

The integration of circumferential electrophysiological mapping with spatial impedance characterization enables tissue identification based not only upon structural properties but also upon functional physiological behavior. As a result, localization accuracy remains substantially improved even in patients exhibiting anatomical variability, tissue thickening, prior surgical alterations, fibrosis, inflammation, or age-related changes in tissue morphology. The combined sensing arrangement further reduces the likelihood of false tissue identification that could occur when relying exclusively on a single sensing modality, while enabling more reliable identification of the target internal anal sphincter region prior to therapeutic deployment. This coordinated acquisition and processing architecture therefore facilitates precise tissue differentiation, enhanced localization confidence, improved procedural consistency, and controlled delivery of therapy to the intended anatomical target.

In an embodiment, the tissue differentiation processor further comprises a tissue profile comparison unit configured to receive acoustic sensing information, electrophysiological sensing information, and impedance sensing information and generate composite tissue characterization information through weighted comparison of multiple tissue parameters including tissue depth characteristics, muscular activity characteristics, and electrical resistance characteristics, and wherein the depth regulation unit further comprises a movable penetration barrier including indexed positioning elements and rotational adjustment structures configured to permit selection of multiple penetration settings corresponding to distinct anatomical dimensions and patient-specific tissue characteristics.

In an embodiment, the tissue differentiation processor incorporates a tissue profile comparison unit configured to perform integrated analysis of multiple forms of tissue response information acquired from the tissue sensing unit in order to generate highly reliable tissue characterization information. The tissue profile comparison unit functions as a multimodal processing engine capable of receiving acoustic sensing information generated by the circumferential acoustic sensing arrangement, electrophysiological sensing information generated by the segmented electrode regions, and impedance sensing information generated by the conductive contact regions. Each sensing modality provides different information relating to the structural, functional, and electrical properties of tissue surrounding the distal insertion portion. The processor therefore combines these complementary datasets to generate a comprehensive representation of the tissue environment rather than relying upon a single sensing parameter that may be influenced by patient-specific anatomical variations.

The acoustic sensing information may include tissue boundary locations, tissue layer thickness measurements, tissue-interface reflection characteristics, acoustic attenuation properties, radial tissue depth values, circumferential continuity measurements, and muscular wall morphology data obtained from reflected ultrasonic signals. Such information enables identification of anatomical structures located at different depths relative to the distal insertion portion. The electrophysiological sensing information may include resting electrical activity levels, spontaneous smooth muscle activation characteristics, electrical conduction behavior, signal amplitude distributions, activation frequencies, circumferential muscular activity patterns, and longitudinal muscular response variations. The impedance sensing information may include tissue resistance values, reactance characteristics, phase angle information, frequency-dependent impedance responses, electrical conductivity distributions, and localized tissue composition indicators. Collectively, these sensing datasets provide a multidimensional representation of tissue structure and physiological behavior.

Upon receiving the sensing information, the tissue profile comparison unit performs a weighted comparison process in which individual tissue parameters are assigned significance values according to their contribution toward tissue identification. The weighting values may be predetermined, dynamically adjustable, or adaptively modified according to signal quality, sensing confidence, patient anatomy, procedural stage, or environmental conditions. For example, when acoustic reflections clearly identify distinct muscular boundaries, greater emphasis may be assigned to tissue depth characteristics. Conversely, when electrical activity measurements exhibit highly distinctive smooth muscle signatures, the processor may increase the weighting associated with muscular activity parameters. Similarly, impedance-derived tissue composition information may receive enhanced weighting when electrical resistance measurements exhibit strong differentiation among adjacent tissue layers.

The processor subsequently performs parameter fusion by correlating tissue depth characteristics with muscular activity characteristics and electrical resistance characteristics to generate composite tissue characterization information. Tissue depth characteristics may indicate the radial position and thickness of a candidate muscular layer, muscular activity characteristics may indicate whether the layer exhibits involuntary smooth muscle behavior or voluntary skeletal muscle behavior, and electrical resistance characteristics may indicate cellular composition and tissue organization. Through simultaneous evaluation of these parameters, the processor identifies tissue regions exhibiting a combination of structural and physiological properties corresponding to the internal anal sphincter. For instance, a tissue layer located at a predetermined depth beneath the mucosal surface, exhibiting continuous circumferential smooth-muscle electrical activity and impedance values associated with dense involuntary muscular tissue, may be classified as the target internal sphincter region with substantially greater confidence than would be achievable using any individual sensing modality.

The tissue profile comparison unit may further generate probabilistic tissue classification models in which each candidate tissue region is assigned a likelihood score corresponding to mucosal tissue, internal sphincter tissue, external sphincter tissue, connective tissue, fibrotic tissue, or other anatomical structures. Such classification may be achieved through rule-based comparison algorithms, statistical correlation models, pattern matching techniques, machine-learning classifiers, neural network architectures, fuzzy logic systems, or combinations thereof. Regions exhibiting the highest composite agreement among the various sensing parameters are selected as verified target locations for subsequent therapeutic deployment. This integrated analysis substantially reduces the influence of isolated sensing artifacts, localized tissue abnormalities, transient physiological changes, and patient-specific anatomical variability.

The depth regulation unit further incorporates a movable penetration barrier configured to provide controlled regulation of penetration depth according to identified anatomical dimensions and patient-specific tissue characteristics. The movable penetration barrier may comprise a mechanical stop assembly positioned within the penetration actuation pathway and configured to physically restrict advancement of the retractable penetration structure beyond a selected deployment distance. The barrier may include indexed positioning elements such as calibrated detents, locking notches, stepped positioning grooves, ratchet engagement structures, numerical depth indicators, or other indexing mechanisms capable of establishing discrete penetration settings. The indexed positioning elements provide repeatable depth selection while preventing inadvertent alteration of the selected setting during a therapeutic procedure.

The movable penetration barrier further includes rotational adjustment structures configured to permit controlled modification of the penetration limit. The rotational adjustment structures may comprise threaded adjustment collars, rotatable depth-control sleeves, screw-based adjustment members, helical positioning mechanisms, gear-driven adjustment assemblies, or cam-operated displacement regulators. Rotation of the adjustment structure translates the barrier longitudinally relative to the penetration actuation pathway, thereby modifying the maximum permissible deployment distance of the retractable penetration structure. The rotational mechanism may further include tactile indexing features, audible detent engagement mechanisms, locking rings, frictional retention assemblies, or position verification indicators to maintain the selected setting throughout the procedure.

Prior to therapeutic deployment, anatomical dimensions identified through tissue characterization may be utilized to determine an appropriate penetration depth. For example, a patient exhibiting a relatively thin internal anal sphincter region may require a shallow penetration setting to ensure localized therapeutic delivery within the target tissue while avoiding extension into adjacent structures. Conversely, a patient exhibiting a thicker muscular layer may require a greater penetration setting to achieve effective delivery within the desired tissue volume. The clinician may therefore adjust the rotational structure until a selected indexed position corresponding to the required penetration depth is achieved. The penetration barrier thereafter mechanically limits advancement of the retractable penetration structure irrespective of continued force applied by the penetration actuation unit.

In an embodiment, the therapeutic delivery unit comprises a hollow penetration structure fluidically coupled to a therapeutic reservoir chamber disposed within the proximal handling portion, and wherein the hollow penetration structure is positioned within a guided translation pathway extending longitudinally through the elongated probe structure and terminating at the deployment opening formed in the distal insertion portion, wherein the penetration actuation unit comprises a spring-biased displacement structure, a guided carriage assembly, and a selectively releasable locking structure configured to convert stored mechanical force into controlled translational displacement of the hollow penetration structure, and wherein the guided carriage assembly constrains movement of the hollow penetration structure along a predetermined deployment axis, and wherein the depth regulation unit comprises an adjustable penetration limiting structure including a threaded stop member positioned adjacent the guided carriage assembly and configured to establish a maximum translational displacement distance corresponding to predefined tissue penetration depths associated with anatomical dimensions of an internal anal sphincter region.

In an embodiment, the therapeutic delivery unit is configured as a mechanically guided therapeutic administration mechanism capable of delivering a therapeutic composition directly into a precisely localized internal anal sphincter region following tissue verification. The therapeutic delivery unit comprises a hollow penetration structure positioned within the elongated probe structure and fluidically coupled to a therapeutic reservoir chamber disposed within the proximal handling portion. The hollow penetration structure may be configured as a fine-gauge injection needle, micro-cannula, tubular penetration conduit, hollow metallic shaft, or reinforced polymeric delivery member fabricated from biocompatible materials such as surgical-grade stainless steel, nitinol, titanium alloys, cobalt-chromium alloys, polyether ether ketone (PEEK), or combinations thereof. The hollow penetration structure includes an internal lumen extending between a proximal fluid inlet and a distal therapeutic discharge opening to facilitate controlled transfer of therapeutic material from the reservoir chamber to the targeted tissue region.

The therapeutic reservoir chamber may comprise a sealed storage compartment configured to contain a predetermined volume of a therapeutic agent intended for administration into the internal anal sphincter. The therapeutic agent may include botulinum toxin formulations, neuromodulatory compounds, regenerative biologics, stem-cell-derived preparations, anti-inflammatory agents, growth factor compositions, localized anesthetics, fibrosis-modulating compounds, muscle relaxation agents, or combinations thereof. The reservoir chamber may further incorporate pressure equalization structures, fluid isolation membranes, collapsible storage bladders, piston-driven displacement mechanisms, or metered dosing components to ensure consistent and controlled delivery of the therapeutic composition. Fluid communication between the reservoir chamber and the hollow penetration structure may be established through microfluidic channels, flexible tubing assemblies, integrated fluid conduits, or sealed transfer pathways configured to maintain sterility and prevent leakage during insertion and deployment.

The hollow penetration structure is positioned within a guided translation pathway extending longitudinally through the elongated probe structure. The guided translation pathway may be formed as an internal channel, linear bore, tubular guide sleeve, reinforced track, or precision-machined passage extending from the proximal handling portion to the deployment opening located at the distal insertion portion. The translation pathway serves to support and align the hollow penetration structure throughout deployment and retraction operations. Internal guide surfaces may include low-friction coatings, lubricious polymer liners, bearing surfaces, or precision alignment features that minimize resistance during movement while maintaining positional accuracy. The deployment opening is dimensioned to permit controlled emergence of the hollow penetration structure while minimizing disturbance to surrounding tissue structures.

The penetration actuation unit is configured to convert stored mechanical energy into controlled advancement of the hollow penetration structure toward the identified target tissue region. The penetration actuation unit includes a spring-biased displacement structure that functions as an energy storage mechanism. The spring-biased displacement structure may incorporate compression springs, extension springs, torsional springs, leaf springs, elastomeric energy storage elements, constant-force springs, or combinations thereof. Prior to deployment, mechanical energy is accumulated within the spring structure while the hollow penetration structure remains secured in a retracted position. The stored energy is maintained until tissue verification procedures confirm the presence of the intended internal anal sphincter region.

The penetration actuation unit further comprises a selectively releasable locking structure configured to retain the spring-biased displacement structure in an energized condition. The locking structure may include latching mechanisms, trigger-actuated retention assemblies, magnetic retention components, mechanical catches, pawl-and-ratchet systems, releasable engagement pins, or electronically controlled release structures. The locking structure may be operatively connected to the deployment authorization processor such that release occurs only after verification criteria have been satisfied. As a result, premature advancement of the hollow penetration structure is prevented during insertion, localization, and tissue differentiation procedures.

Upon receipt of a deployment authorization signal, the locking structure disengages and permits controlled release of stored mechanical energy from the spring-biased displacement structure. The released energy is transferred to a guided carriage assembly mechanically coupled to the hollow penetration structure. The guided carriage assembly may comprise a sliding carriage, linear shuttle mechanism, rail-guided support platform, bearing-mounted translation member, or precision alignment carrier configured to move along the guided translation pathway. The carriage assembly maintains positional stability of the hollow penetration structure throughout deployment and prevents undesired lateral displacement, rotational deviation, bending, buckling, or off-axis movement that could compromise targeting accuracy.

The guided carriage assembly constrains movement of the hollow penetration structure along a predetermined deployment axis extending substantially longitudinally through the elongated probe structure. Such constrained movement ensures that advancement occurs in a predictable and repeatable manner toward the deployment opening. In certain implementations, the carriage assembly may include guide rails, alignment grooves, sliding bearings, anti-rotation features, stabilizing bushings, or linear guide elements that continuously maintain axial alignment throughout the deployment sequence. The resulting controlled translational displacement enables the penetration structure to enter tissue at a predetermined location and orientation corresponding to the verified target region.

The depth regulation unit further comprises an adjustable penetration limiting structure configured to establish a maximum allowable advancement distance for the hollow penetration structure. The penetration limiting structure may include a threaded stop member positioned adjacent to the guided carriage assembly and mechanically coupled to an adjustment mechanism accessible from the proximal handling portion. The threaded stop member may comprise a threaded collar, adjustable screw stop, helical depth regulator, rotatable depth-control sleeve, threaded shaft assembly, or calibrated adjustment member capable of modifying the permissible travel distance of the carriage assembly. Rotation of the threaded stop member causes longitudinal displacement of the stop position due to engagement between complementary threaded surfaces.

The threaded stop member may further incorporate graduated markings, indexed depth indicators, tactile positioning detents, locking mechanisms, or calibration references corresponding to predefined tissue penetration depths. Prior to deployment, the clinician may select an appropriate penetration setting based upon anatomical information generated by the tissue differentiation processor. For example, if tissue characterization indicates that the internal anal sphincter is located approximately 4 millimeters beneath the mucosal interface, the threaded stop member may be adjusted to permit advancement corresponding to that depth. Similarly, in a patient exhibiting a thicker sphincter region located at a greater radial distance from the probe surface, the threaded stop member may be repositioned to allow increased penetration distance.

During deployment, the guided carriage assembly advances under the force generated by the spring-biased displacement structure until physical engagement occurs between the carriage assembly and the threaded stop member. Upon contact, further movement is mechanically restricted irrespective of any residual force remaining within the actuation mechanism. Consequently, the penetration depth remains confined to the predetermined value established through adjustment of the penetration limiting structure. This mechanical limitation provides an additional level of safety independent of software-based controls and ensures reproducible penetration depths across multiple procedures.

In an embodiment, the signal acquisition processor is configured to sequentially activate individual high-frequency ultrasound transducer elements of the circumferential acoustic sensing arrangement disposed within the tissue sensing unit, receive reflected acoustic signals corresponding to a plurality of circumferential tissue locations surrounding the distal insertion portion, generate acoustic response information representative of tissue-interface positions at the plurality of circumferential tissue locations, and transmit the acoustic response information to the tissue differentiation processor, and wherein the tissue differentiation processor is configured to compare tissue-interface positions identified from the plurality of circumferential tissue locations to determine continuity of a muscular tissue boundary surrounding the distal insertion portion and generate the tissue characterization information based upon the determined continuity of the muscular tissue boundary.

In an embodiment, the tissue sensing unit incorporates a circumferential acoustic sensing arrangement comprising a plurality of high-frequency ultrasound transducer elements distributed around the outer periphery of the distal insertion portion. The transducer elements are configured to generate and receive ultrasonic energy for real-time evaluation of tissue structures surrounding the elongated probe structure. Each transducer element may comprise a piezoelectric crystal, piezocomposite material, capacitive micromachined ultrasonic transducer (CMUT), microelectromechanical ultrasonic transducer, or equivalent acoustic energy conversion device capable of operating at frequencies selected to provide high-resolution visualization of soft tissue interfaces. In certain implementations, the transducer elements may operate within a frequency range of approximately 10 MHz to 50 MHz, enabling detailed assessment of muscular layers, mucosal boundaries, connective tissue structures, and adjacent anatomical features within the anal canal.

The signal acquisition processor is configured to selectively and sequentially activate individual ultrasound transducer elements according to a predetermined scanning sequence. Rather than simultaneously energizing all transducer elements, the processor activates each element independently to obtain localized acoustic information from discrete circumferential tissue locations. During activation, a selected transducer element emits a controlled ultrasonic pulse into the surrounding tissue environment. The emitted acoustic energy propagates radially outward from the distal insertion portion and encounters various tissue structures exhibiting different acoustic impedances. Whenever the propagating acoustic wave encounters an interface between tissues having differing densities, elastic properties, hydration levels, or structural compositions, a portion of the acoustic energy is reflected back toward the transducer element.

The signal acquisition processor receives the reflected acoustic signals and converts the received echoes into digital information suitable for further analysis. Signal conditioning operations may include amplification, filtering, noise suppression, gain compensation, time-gain correction, analog-to-digital conversion, and echo envelope detection. The processor then determines the time interval between acoustic pulse transmission and echo reception to calculate distances between the transducer element and detected tissue interfaces. Since the propagation velocity of ultrasonic energy through biological tissue is known or can be estimated, the processor converts the measured travel times into radial tissue depth measurements corresponding to individual circumferential sensing locations.

As the sequential activation process progresses around the circumference of the distal insertion portion, the processor acquires reflected acoustic information from multiple angular positions surrounding the probe. The resulting dataset contains tissue-interface position information corresponding to a plurality of circumferential tissue locations. Such tissue-interface positions may represent boundaries between mucosal tissue and submucosal tissue, interfaces between connective tissue and muscular tissue, transitions between smooth muscle and skeletal muscle structures, or interfaces associated with fibrotic regions, lesions, or other anatomical features. The processor organizes the acquired measurements according to circumferential position and generates acoustic response information representing the spatial arrangement of tissue layers surrounding the probe.

The acoustic response information is subsequently transmitted to the tissue differentiation processor for advanced tissue characterization. The tissue differentiation processor receives the tissue-interface positions identified at the plurality of circumferential sensing locations and performs comparative analysis to determine geometric relationships among the detected tissue boundaries. More specifically, the processor evaluates whether identified tissue interfaces form a substantially continuous circumferential structure surrounding the distal insertion portion. Such continuity analysis is particularly useful for identifying muscular tissue layers that typically exhibit annular or circumferential anatomical configurations within the anal canal.

The continuity determination process may involve comparison of radial depth measurements obtained from neighboring circumferential sensing locations. Where tissue-interface positions exhibit gradual variation and maintain spatial consistency around the circumference of the probe, the processor may classify the detected boundary as a continuous muscular layer. Conversely, abrupt discontinuities, interruptions, irregular depth transitions, or localized distortions may indicate the presence of non-muscular structures, anatomical abnormalities, scar tissue, tissue defects, or regions outside the intended target anatomy. The processor may further calculate continuity metrics including circumferential completeness values, boundary uniformity parameters, radial variance measurements, geometric symmetry indicators, and spatial coherence coefficients to quantify the degree of continuity exhibited by the detected tissue layer.

In certain implementations, the tissue differentiation processor may construct a circumferential tissue boundary model by connecting tissue-interface positions identified at successive circumferential sensing locations. The resulting model enables visualization of the shape, thickness, and continuity of the detected muscular structure. For example, a healthy internal anal sphincter typically forms a substantially continuous circumferential ring of smooth muscle surrounding the anal canal. When acoustic response information identifies a tissue boundary exhibiting a continuous annular geometry with relatively uniform radial positioning, the processor may classify the boundary as corresponding to a muscular tissue layer associated with the internal anal sphincter. In contrast, fragmented boundary patterns or irregular interface distributions may indicate adjacent tissue regions that do not correspond to the intended therapeutic target.

The tissue differentiation processor may further incorporate pattern recognition algorithms configured to compare the detected circumferential boundary characteristics against stored anatomical reference profiles. Such reference profiles may contain expected boundary geometries, thickness distributions, radial positions, and continuity characteristics associated with mucosal tissue layers, internal sphincter tissue, external sphincter tissue, and other surrounding anatomical structures. By comparing acquired acoustic response information with the stored reference profiles, the processor can determine the degree of correspondence between the detected tissue layer and known anatomical structures. This comparison improves localization reliability and reduces the possibility of tissue misidentification resulting from patient-specific anatomical variations.

In an exemplary implementation, the distal insertion portion may be positioned within the anal canal such that individual transducer elements sequentially acquire acoustic information from thirty-two or more circumferential sensing locations. The processor may determine that a particular tissue boundary is detected at substantially similar radial distances around the entire circumference of the probe, thereby indicating the presence of a continuous muscular ring. Simultaneously, continuity analysis may reveal that the boundary exhibits minimal radial deviation and high circumferential completeness. Based upon these characteristics, the tissue differentiation processor may identify the detected structure as corresponding to the internal anal sphincter region. If a localized interruption in the boundary is observed, the processor may continue acquisition of additional sensing cycles until sufficient continuity confidence is achieved.

The described arrangement enables tissue characterization to be based not merely upon isolated tissue depth measurements but upon comprehensive assessment of circumferential tissue architecture. By evaluating the continuity of muscular boundaries surrounding the distal insertion portion, the system can distinguish anatomically significant muscular structures from isolated tissue interfaces or transient sensing artifacts. The resulting characterization process improves localization accuracy, enhances recognition of circumferential muscular anatomy, reduces sensitivity to localized measurement errors, supports consistent identification of target tissue regions across different patients, and provides a more reliable basis for subsequent therapeutic deployment within the identified internal anal sphincter region.

In an embodiment, the signal acquisition processor is configured to acquire electrical activity information from the annular conductive sensing elements of the electrophysiological sensing arrangement during a plurality of successive acquisition intervals occurring while the elongated probe structure is positioned within the anal canal, and wherein the tissue differentiation processor is configured to organize the electrical activity information according to circumferential sensing location, determine spatial variation of muscular activity surrounding the distal insertion portion, compare the spatial variation of muscular activity with acoustic response information generated from the circumferential acoustic sensing arrangement, and identify tissue characteristics corresponding to the internal anal sphincter region from a spatial correlation between the muscular activity information and the acoustic response information.

The signal acquisition processor is configured to acquire electrical activity information from the annular conductive sensing elements during a plurality of successive acquisition intervals while the elongated probe structure remains positioned within the anal canal. Each acquisition interval may correspond to a predefined sampling period during which electrical signals generated by surrounding muscular tissue are detected, amplified, conditioned, and digitized. The successive acquisition intervals enable continuous monitoring of tissue behavior over time and allow the processor to capture dynamic physiological activity rather than relying upon isolated measurements. Such repeated acquisition improves robustness against transient signal fluctuations, motion-related artifacts, temporary muscular contractions, and environmental electrical noise.

The acquired electrical activity information may include resting bioelectrical potentials, spontaneous muscular activation signals, neuromuscular conduction patterns, signal amplitude distributions, activation frequencies, waveform morphology, temporal firing characteristics, propagation behavior, and circumferential activation sequences associated with surrounding tissue structures. Because different anatomical tissues exhibit distinct electrophysiological behavior, the collected electrical activity information provides insight into both tissue composition and physiological function. For example, smooth muscle tissue associated with the internal anal sphincter generally demonstrates sustained involuntary electrical activity characterized by relatively stable activation patterns, whereas skeletal muscle tissue associated with the external anal sphincter may exhibit intermittent activation patterns influenced by voluntary neuromuscular control.

Following acquisition, the tissue differentiation processor receives the electrical activity information and organizes the acquired data according to circumferential sensing location. More specifically, electrical signals acquired from individual conductive sensing elements are associated with corresponding angular positions around the distal insertion portion. This spatial organization enables the processor to evaluate how muscular activity varies around the circumference of the probe rather than treating all acquired signals as a single aggregate measurement. The processor may generate circumferential activity maps, angular activity distributions, localized activation profiles, and spatially resolved electrophysiological representations corresponding to the surrounding tissue environment.

The tissue differentiation processor subsequently determines spatial variation of muscular activity surrounding the distal insertion portion. Such spatial variation may be evaluated through comparison of signal amplitudes, activation frequencies, waveform characteristics, electrical conduction properties, synchronization behavior, and temporal response patterns observed at different circumferential sensing locations. Regions exhibiting relatively uniform electrical activity around the circumference may indicate the presence of a continuous muscular structure, whereas abrupt changes in activity between adjacent sensing locations may indicate tissue transitions, localized anatomical variations, muscular defects, fibrotic regions, or boundaries between different tissue layers.

In addition to electrophysiological analysis, the tissue differentiation processor receives acoustic response information generated from the circumferential acoustic sensing arrangement. The acoustic response information may include tissue-interface positions, tissue layer thickness measurements, radial depth values, muscular boundary locations, acoustic reflection characteristics, tissue continuity indicators, and circumferential anatomical profiles determined from ultrasonic sensing operations. Because the acoustic sensing arrangement provides structural information while the electrophysiological sensing arrangement provides functional information, the processor is capable of evaluating tissue characteristics using complementary datasets that describe both anatomy and physiological behavior.

The tissue differentiation processor compares the spatial variation of muscular activity with the acoustic response information through spatial correlation procedures. In one implementation, the processor aligns electrophysiological activity maps with corresponding acoustic tissue maps according to circumferential position and radial location. The processor then evaluates whether regions exhibiting characteristic muscular electrical activity correspond to anatomical structures identified through acoustic sensing. For example, a circumferential tissue layer identified acoustically at a particular radial depth may be compared with electrical activity measurements obtained from sensing locations adjacent to that layer. Where the anatomical structure identified acoustically coincides with a region exhibiting characteristic smooth-muscle electrical behavior, the processor may determine that the structure corresponds to the internal anal sphincter.

The spatial correlation process may further involve generation of correlation coefficients, overlap measurements, confidence scores, pattern matching outputs, similarity indices, probabilistic classifications, or anatomical correspondence values. The processor may evaluate whether areas of elevated smooth-muscle activity coincide with acoustically identified continuous muscular boundaries and whether the circumferential distribution of electrical activity matches the geometry of the detected tissue layer. A high degree of correspondence between structural and functional information provides strong evidence that the identified tissue region represents the intended therapeutic target.

In an exemplary implementation, the acoustic sensing arrangement may identify a circumferential tissue layer located approximately four millimeters beneath the mucosal surface and extending substantially around the circumference of the anal canal. Simultaneously, the electrophysiological sensing arrangement may detect relatively uniform involuntary muscular activity at circumferential sensing locations corresponding to the identified layer. The tissue differentiation processor may spatially align the acoustic boundary data and the muscular activity distribution data and determine that the areas of greatest smooth-muscle activity coincide with the acoustically detected muscular structure. Based upon this spatial correspondence, the processor may classify the identified region as the internal anal sphincter with a high degree of confidence.

In another example, acoustic sensing may identify multiple tissue interfaces located at different radial depths. While some interfaces may correspond to connective tissue layers or mucosal structures, only one of the identified boundaries may exhibit a strong spatial relationship with circumferential smooth-muscle electrical activity. Through correlation analysis, the processor is able to distinguish the actual internal sphincter from adjacent anatomical structures that may possess similar geometric characteristics but lack the expected physiological activity profile. This capability is particularly advantageous in patients exhibiting anatomical variability, muscular hypertrophy, fibrosis, inflammation, or prior surgical modifications.

In an embodiment, the tissue differentiation processor is configured to receive acoustic response information generated from the circumferential acoustic sensing arrangement and electrical activity information generated from the electrophysiological sensing arrangement, determine a plurality of candidate tissue transition locations from the acoustic response information, determine a plurality of muscular tissue locations from the electrical activity information, compare the candidate tissue transition locations with the muscular tissue locations, and generate the tissue characterization information only after identification of a spatial relationship between the candidate tissue transition locations and the muscular tissue locations corresponding to an internal anal sphincter tissue profile.

The circumferential acoustic sensing arrangement continuously acquires acoustic response information from tissue structures surrounding the distal insertion portion. The acoustic response information may include tissue-interface positions, reflection intensities, acoustic attenuation characteristics, tissue layer thickness values, boundary continuity information, radial depth measurements, and circumferential tissue morphology data. Following acquisition, the tissue differentiation processor analyzes the acoustic response information to identify locations where significant changes in tissue properties occur. Such locations may correspond to boundaries between mucosal tissue and muscular tissue, transitions between smooth muscle and skeletal muscle structures, connective tissue interfaces, fibrotic regions, or other anatomical layer changes. These identified regions are designated as candidate tissue transition locations because they represent potential anatomical boundaries that may correspond to the target internal anal sphincter region.

The determination of candidate tissue transition locations may be performed through analysis of abrupt changes in acoustic impedance, variations in reflected signal amplitude, alterations in tissue layer thickness, discontinuities in tissue-interface geometry, or changes in circumferential tissue morphology. The processor may employ edge detection algorithms, gradient analysis techniques, pattern recognition procedures, tissue boundary extraction algorithms, segmentation models, or machine-learning-based tissue classification methods to identify such transition locations. In certain implementations, the processor may assign a confidence value to each candidate transition location according to the strength and consistency of the detected acoustic features.

Simultaneously, the electrophysiological sensing arrangement acquires electrical activity information from tissue surrounding the distal insertion portion. The acquired electrical activity information may include resting electrical potentials, spontaneous muscular activation signals, signal amplitude characteristics, conduction patterns, activation frequencies, waveform morphology, synchronization behavior, and temporal electrical response characteristics. The tissue differentiation processor analyzes these signals to identify locations exhibiting characteristics associated with muscular tissue. Such locations are designated as muscular tissue locations and represent regions where electrical activity patterns indicate the presence of active muscle fibers.

The determination of muscular tissue locations may involve evaluating signal intensity, activation consistency, circumferential electrical distribution, frequency-domain characteristics, conduction behavior, and temporal activation patterns. Regions exhibiting continuous involuntary muscular activity characteristic of smooth muscle tissue may be distinguished from regions exhibiting voluntary skeletal muscle activity or regions exhibiting minimal electrical activity associated with connective or mucosal tissues. The processor may generate circumferential muscular activity maps and longitudinal activity profiles that identify the spatial distribution of muscular structures surrounding the probe.

After determining the candidate tissue transition locations and the muscular tissue locations, the tissue differentiation processor performs a comparative spatial analysis to evaluate relationships between the two datasets. More specifically, the processor determines whether anatomical transition regions identified from acoustic sensing correspond spatially with regions exhibiting muscular activity identified from electrophysiological sensing. The comparison may involve alignment of circumferential coordinates, radial depth positions, longitudinal locations, angular distributions, and tissue boundary geometries obtained from both sensing modalities. The processor may calculate overlap values, spatial correspondence metrics, correlation coefficients, proximity measurements, and anatomical matching scores to quantify the relationship between the identified locations.

In one implementation, a candidate tissue transition location identified through acoustic sensing may correspond to a circumferential boundary separating a superficial tissue layer from a deeper muscular layer. The processor subsequently evaluates whether the deeper layer exhibits electrical activity characteristics associated with smooth muscle tissue. If the identified muscular activity region is spatially adjacent to or coincident with the candidate transition location, the processor may determine that the observed relationship corresponds to a transition into an internal sphincter structure. Conversely, if the candidate transition location does not correspond with an appropriate muscular activity profile, the processor may reject the location as a valid target despite its acoustic appearance.

The tissue differentiation processor may further compare the detected spatial relationship against stored internal anal sphincter tissue profiles maintained within a reference database. Such tissue profiles may define expected anatomical and physiological characteristics including radial depth ranges, circumferential continuity patterns, smooth muscle activity signatures, tissue layer thickness distributions, impedance characteristics, and tissue transition geometries associated with the internal anal sphincter. The processor evaluates whether the observed relationship between candidate transition locations and muscular tissue locations satisfies the predefined criteria associated with the stored tissue profile.

For example, an internal anal sphincter tissue profile may specify the presence of a substantially continuous circumferential muscular layer located beneath a mucosal boundary, wherein the muscular layer exhibits uniform smooth-muscle electrical activity around a substantial portion of the circumference. If acoustic sensing identifies a circumferential tissue boundary at a depth of approximately four millimeters and electrophysiological sensing simultaneously identifies continuous involuntary muscular activity immediately adjacent to that boundary, the processor may determine that the observed spatial relationship corresponds to the stored internal sphincter profile. Tissue characterization information is subsequently generated to indicate successful identification of the target tissue region.

In an embodiment, the deployment authorization processor is configured to receive a first tissue characterization information set generated during a first sensing cycle, receive a second tissue characterization information set generated during a subsequent sensing cycle, compare the first tissue characterization information set and the second tissue characterization information set to determine positional consistency of an identified internal anal sphincter region, maintain restriction of the penetration actuation unit while the positional consistency remains below a predetermined verification condition, and permit operation of the penetration actuation unit after the positional consistency satisfies the predetermined verification condition.

During operation, the tissue sensing unit performs a first sensing cycle while the elongated probe structure is positioned within the anal canal. The first sensing cycle may involve acquisition of acoustic response information, electrophysiological sensing information, impedance information, or combinations thereof. The tissue differentiation processor analyzes the acquired information and generates a first tissue characterization information set representing the location, depth, circumferential position, tissue profile characteristics, confidence metrics, boundary continuity parameters, muscular activity distributions, and anatomical attributes associated with a candidate internal anal sphincter region. The first tissue characterization information set is subsequently transmitted to the deployment authorization processor and temporarily stored within a memory structure associated with the processor.

Following completion of the first sensing cycle, the tissue sensing unit performs a subsequent sensing cycle while the elongated probe structure remains positioned within the anatomical region of interest. The subsequent sensing cycle may occur immediately after the first sensing cycle or after a predetermined interval sufficient to acquire additional tissue response information. During the subsequent sensing cycle, new acoustic measurements, electrophysiological measurements, and optional impedance measurements are obtained and processed by the tissue differentiation processor to generate a second tissue characterization information set. The second tissue characterization information set contains corresponding localization information describing the tissue region identified during the subsequent sensing operation.

The deployment authorization processor receives the second tissue characterization information set and performs a comparative analysis between the first tissue characterization information set and the second tissue characterization information set. The comparison process may involve evaluation of radial tissue depth measurements, circumferential location coordinates, longitudinal tissue positions, muscular activity distributions, tissue-interface locations, tissue boundary geometries, confidence values, anatomical profile characteristics, and other localization parameters. The processor determines whether the tissue region identified during the subsequent sensing cycle corresponds substantially to the tissue region identified during the preceding sensing cycle.

To quantify this relationship, the processor determines a positional consistency value representing the degree of spatial agreement between the identified tissue locations. The positional consistency value may be calculated using coordinate comparison algorithms, overlap calculations, spatial correlation methods, geometric matching procedures, statistical similarity analyses, probabilistic localization models, or machine-learning-based consistency evaluation techniques. In certain implementations, positional consistency may be represented as a numerical score indicating the degree of correspondence between the tissue locations identified during successive sensing cycles.

For example, if the first tissue characterization information set identifies an internal anal sphincter boundary located at a radial depth of approximately four millimeters and extending through a particular circumferential region, and the second tissue characterization information set identifies a substantially identical boundary at nearly the same location, the processor may determine that a high degree of positional consistency exists. Conversely, if the second sensing cycle identifies a tissue region exhibiting substantial displacement, altered geometry, inconsistent muscular activity patterns, or significantly different anatomical characteristics, the processor may determine that the positional consistency remains inadequate for reliable therapeutic targeting.

The processor compares the determined positional consistency against a predetermined verification condition. The verification condition may comprise a minimum spatial overlap threshold, a maximum allowable positional deviation, a minimum correlation coefficient, a required confidence level, a predefined geometric similarity value, or a combination of multiple verification criteria. The verification condition may be established according to clinical requirements, device calibration parameters, patient-specific anatomical considerations, or procedural safety protocols. In certain embodiments, the verification condition may require that localization measurements remain substantially unchanged over multiple consecutive sensing cycles before deployment authorization is granted.

While the positional consistency remains below the predetermined verification condition, the deployment authorization processor maintains restriction of the penetration actuation unit. Such restriction may be achieved through electronic disabling of an actuation trigger, maintenance of a mechanical locking structure in an engaged condition, interruption of power supplied to an electromechanical actuator, prevention of release of a spring-biased deployment mechanism, or activation of a software-controlled interlock. Consequently, even if an operator attempts to initiate deployment, advancement of the penetration structure remains prohibited until sufficient localization consistency has been verified.

In certain implementations, the processor may continue to request additional sensing cycles while the verification condition remains unsatisfied. Each newly generated tissue characterization information set may be compared with one or more previously acquired information sets to refine the positional consistency assessment. As additional sensing information becomes available, the processor may continuously update the consistency calculation and determine whether localization confidence improves over time. This repeated verification process enables rejection of transient localization events that may arise from involuntary muscular contractions, patient movement, probe repositioning, temporary signal interference, or localized sensing abnormalities.

In an exemplary clinical scenario, a first sensing cycle may identify an internal anal sphincter region at a circumferential position corresponding to a particular angular location around the distal insertion portion. A second sensing cycle acquired shortly thereafter may identify substantially the same tissue boundary with only minimal positional deviation. The deployment authorization processor may determine that the positional consistency exceeds a predefined acceptance threshold and therefore conclude that the identified tissue location is stable and reproducible. Upon satisfaction of the verification condition, the processor generates a deployment authorization signal and permits operation of the penetration actuation unit.

In another example, the first sensing cycle may identify a candidate tissue region that appears consistent with an internal anal sphincter profile. However, during the subsequent sensing cycle, the identified tissue location may shift significantly due to temporary probe displacement or transient tissue movement. The resulting positional consistency value may fall below the predetermined verification condition. In response, the deployment authorization processor continues to maintain restriction of the penetration actuation unit and requests additional sensing information until a stable tissue location is repeatedly confirmed. As a result, therapeutic deployment is prevented until adequate localization certainty is achieved.

By requiring confirmation that tissue localization remains consistent across multiple sensing cycles, the deployment authorization processor introduces an additional verification layer that supplements the tissue differentiation process. This approach reduces susceptibility to isolated sensing errors, transient physiological variations, temporary anatomical distortions, and operator-dependent positioning inconsistencies. The resulting control architecture improves reliability of tissue targeting, enhances procedural reproducibility, minimizes the likelihood of unintended therapeutic delivery, and provides a robust mechanism for ensuring that therapeutic deployment occurs only after repeated confirmation of the identified internal anal sphincter region.

In an embodiment, the deployment authorization processor is configured to receive tissue characterization information corresponding to a plurality of circumferential sensing locations surrounding the distal insertion portion, determine whether tissue characteristics corresponding to the internal anal sphincter region are detected at multiple circumferential sensing locations, generate a circumferential verification result from the detected tissue characteristics, and permit extension of the retractable penetration structure only after the circumferential verification result satisfies a predefined tissue-localization condition.

During operation, the tissue differentiation processor generates tissue characterization information corresponding to a plurality of circumferential sensing locations surrounding the distal insertion portion. These sensing locations may be established through circumferentially distributed ultrasound transducer elements, segmented electrophysiological sensing elements, impedance sensing contacts, or combinations thereof. Each circumferential sensing location produces localized information relating to tissue depth, tissue-interface position, muscular activity characteristics, electrical impedance properties, tissue continuity measurements, and anatomical boundary information. The resulting tissue characterization information therefore represents a circumferential profile of tissue conditions surrounding the probe rather than a single-point assessment.

The deployment authorization processor receives the tissue characterization information generated from the various circumferential sensing locations and organizes the information according to angular position around the distal insertion portion. The processor may divide the circumference into multiple sectors, zones, or sensing segments, each corresponding to a particular circumferential region. For example, the circumference may be represented as a series of angular sectors extending through 360 degrees around the distal insertion portion. Tissue characteristics identified within each sector are individually evaluated and subsequently compared with characteristics identified in neighboring sectors.

The processor then determines whether tissue characteristics corresponding to the internal anal sphincter region are detected at multiple circumferential sensing locations. Such tissue characteristics may include identification of a smooth-muscle tissue layer at an expected radial depth, presence of continuous circumferential muscular boundaries, electrophysiological activity patterns associated with involuntary smooth muscle tissue, impedance characteristics corresponding to sphincter musculature, and acoustic signatures indicative of a circumferential muscular structure. Detection of these characteristics across multiple circumferential locations provides evidence that the identified tissue region forms part of a continuous anatomical structure rather than an isolated tissue feature.

The determination process may involve evaluating the extent to which the identified tissue characteristics are distributed around the circumference of the distal insertion portion. The processor may calculate circumferential coverage values, continuity measurements, angular distribution parameters, sector occupancy values, boundary completeness indicators, or tissue correspondence metrics. For example, if tissue characteristics associated with the internal anal sphincter are detected across a substantial proportion of the circumferential sensing locations, the processor may determine that the identified tissue exhibits the continuity expected of a sphincter structure. Conversely, if such characteristics are detected only within a limited angular region, the processor may determine that the identified tissue does not satisfy the expected anatomical profile.

Following analysis of the circumferential distribution of tissue characteristics, the deployment authorization processor generates a circumferential verification result. The circumferential verification result may comprise a numerical score, confidence value, classification output, continuity index, verification status indicator, probability estimate, or composite verification parameter representing the degree to which the detected tissue characteristics correspond to a circumferential internal anal sphincter profile. The verification result may incorporate information relating to tissue continuity, circumferential completeness, anatomical symmetry, muscular activity distribution, structural consistency, and multimodal sensing agreement.

In certain implementations, the circumferential verification result is generated through comparison of acquired tissue information with one or more stored anatomical reference models. The reference models may define expected circumferential characteristics of the internal anal sphincter, including expected radial positions, tissue thickness ranges, continuity requirements, smooth-muscle activity distributions, and circumferential geometric relationships. The processor compares the acquired sensing information against the reference models and determines the degree of correspondence between the detected tissue structure and the expected anatomical profile. Greater correspondence results in a higher circumferential verification result and greater confidence in tissue localization.

The deployment authorization processor subsequently compares the circumferential verification result against a predefined tissue-localization condition. The predefined tissue-localization condition may include a minimum circumferential coverage requirement, a minimum continuity threshold, a required verification score, a minimum confidence level, a predetermined anatomical correspondence value, or a combination of multiple acceptance criteria. The predefined condition may be established during device calibration, according to clinical treatment protocols, or based upon patient-specific procedural requirements.

Until the circumferential verification result satisfies the predefined tissue-localization condition, the deployment authorization processor maintains restriction of the retractable penetration structure. Such restriction may be implemented through maintenance of a mechanical locking assembly, prevention of release of a spring-biased deployment mechanism, deactivation of an electromechanical actuator, interruption of deployment control signals, or activation of an electronic interlock mechanism. As a result, extension of the retractable penetration structure remains prohibited despite identification of a potential target tissue region.

When the circumferential verification result satisfies the predefined tissue-localization condition, the processor determines that sufficient evidence exists to confirm localization of the internal anal sphincter region. The deployment authorization processor then generates a deployment authorization signal permitting extension of the retractable penetration structure toward the identified target tissue. Because authorization is based upon confirmation of circumferential tissue characteristics rather than isolated sensing data, the likelihood of erroneous deployment into adjacent tissues is substantially reduced.

In an exemplary implementation, circumferential sensing data may be acquired from thirty-two sensing sectors distributed around the distal insertion portion. The tissue differentiation processor may identify tissue characteristics associated with smooth-muscle sphincter tissue in twenty-eight of the thirty-two sectors. The deployment authorization processor calculates a circumferential coverage value indicating that the identified tissue extends through a substantial portion of the circumference and exhibits continuity consistent with a sphincter structure. Because the calculated verification result exceeds the predefined tissue-localization condition, deployment authorization is granted. In contrast, if similar tissue characteristics are detected in only a small number of sectors, the processor determines that the circumferential verification requirement has not been satisfied and maintains deployment restriction until additional sensing confirms the target tissue location.

By requiring verification of tissue characteristics across multiple circumferential sensing locations, the deployment authorization processor introduces an additional anatomical validation mechanism that complements the structural and functional tissue differentiation processes. This verification approach improves localization reliability, reduces the influence of localized sensing artifacts, accommodates anatomical variability among patients, minimizes the probability of false-positive tissue identification, and ensures that therapeutic deployment is performed only after confirmation that the detected tissue exhibits the circumferential characteristics expected of the internal anal sphincter region.

In an embodiment, the tissue differentiation processor is configured to generate a circumferential tissue map by associating acoustic response information acquired from the circumferential acoustic sensing arrangement with electrical activity information acquired from the electrophysiological sensing arrangement at corresponding circumferential sensing locations, identify a tissue transition zone separating a smooth muscle region from an adjacent tissue region within the circumferential tissue map, and generate the tissue characterization information from the identified tissue transition zone.

During operation, the circumferential acoustic sensing arrangement acquires acoustic response information from a plurality of circumferential sensing locations distributed around the distal insertion portion. The acoustic response information may include tissue-interface positions, tissue boundary locations, radial depth measurements, tissue layer thickness values, acoustic impedance variations, reflection intensity characteristics, attenuation profiles, and circumferential continuity measurements. The signal acquisition processor organizes the acquired information according to angular position around the circumference of the probe and generates spatially resolved structural data corresponding to surrounding anatomical layers. As the probe advances or remains stationary within the anal canal, multiple acoustic measurements may be acquired to improve resolution and reduce the influence of transient measurement artifacts.

Simultaneously, the electrophysiological sensing arrangement acquires electrical activity information from corresponding circumferential sensing locations. The acquired electrical activity information may include resting electrical potentials, spontaneous muscular activation signals, signal amplitudes, conduction characteristics, frequency-domain parameters, waveform morphology, temporal activation sequences, and localized neuromuscular response patterns. The signal acquisition processor associates the electrical activity information with the corresponding circumferential positions from which the signals were acquired, thereby enabling direct comparison between structural and functional tissue characteristics at common sensing locations.

The tissue differentiation processor receives both datasets and performs spatial registration of the acoustic response information and the electrical activity information. Spatial registration may involve alignment of sensing coordinates, angular positions, radial depth references, longitudinal locations, and circumferential sector identifiers such that structural and functional measurements obtained from corresponding regions are accurately correlated. Following alignment, the processor generates the circumferential tissue map by assigning acoustic characteristics and electrophysiological characteristics to corresponding circumferential locations surrounding the distal insertion portion. The resulting map may comprise a two-dimensional or three-dimensional representation in which radial distance, circumferential position, tissue depth, tissue boundaries, and muscular activity distributions are simultaneously represented.

Within the generated circumferential tissue map, the processor evaluates relationships between structural features and physiological activity patterns to identify tissue transition zones. A tissue transition zone may correspond to a region where one tissue type changes into another tissue type, such as a transition between mucosal tissue and smooth muscle tissue, a transition between smooth muscle tissue and skeletal muscle tissue, or a transition between muscular tissue and surrounding connective tissue structures. Such transition zones frequently represent clinically significant anatomical boundaries that are useful for localization of the internal anal sphincter.

The identification of tissue transition zones may be performed through detection of concurrent changes in structural and electrophysiological characteristics. For example, the processor may identify a location where acoustic sensing indicates the presence of a new circumferential tissue layer at a particular radial depth while electrophysiological sensing simultaneously indicates the onset of sustained smooth-muscle electrical activity. The coincidence of these structural and functional changes may be interpreted as a transition from a non-muscular tissue layer into a smooth muscle region. Similarly, a reduction in smooth-muscle electrical activity accompanied by a change in tissue-interface geometry may indicate transition into a different anatomical structure.

The processor may employ segmentation algorithms, edge-detection procedures, gradient analysis methods, pattern recognition techniques, probabilistic classification models, machine-learning frameworks, or anatomical reference matching procedures to identify such transition zones. In certain implementations, tissue transition zones are characterized by abrupt changes in acoustic reflection intensity, alterations in tissue thickness, variations in radial tissue depth, shifts in muscular activity amplitude, changes in signal frequency characteristics, or modifications in conduction behavior. By evaluating these parameters collectively rather than independently, the processor achieves a more comprehensive understanding of tissue composition and anatomical organization.

In an embodiment, the deployment authorization processor is configured to receive the tissue characterization information from the tissue differentiation processor during a plurality of successive sensing cycles, determine a localization stability value according to variation in tissue characteristics identified during the plurality of successive sensing cycles, maintain the penetration actuation unit in a deployment-restricted condition while the localization stability value varies beyond a predetermined range, and permit operation of the penetration actuation unit after the localization stability value remains within the predetermined range throughout a plurality of consecutive sensing cycles.

In an embodiment, the deployment authorization processor is configured to evaluate the temporal stability of tissue localization before permitting therapeutic deployment. The processor operates on the principle that a correctly identified internal anal sphincter region should exhibit substantially consistent localization characteristics across multiple sensing cycles acquired over time. Localized tissue measurements obtained during a single sensing event may occasionally be influenced by transient physiological conditions, involuntary muscular contractions, probe movement, patient motion, temporary acoustic artifacts, electrical noise, or localized tissue deformation. Accordingly, the deployment authorization processor continuously monitors tissue characterization information generated during a plurality of successive sensing cycles and determines whether the identified tissue region remains stable over an extended verification period before authorizing activation of the penetration actuation unit.

During operation, the tissue sensing unit repeatedly acquires tissue response information while the elongated probe structure remains positioned within the anal canal. Each sensing cycle may involve acquisition of acoustic response information, electrophysiological activity information, impedance sensing information, or combinations thereof. The tissue differentiation processor analyzes the acquired information during each sensing cycle and generates corresponding tissue characterization information identifying one or more candidate tissue regions, associated tissue boundaries, muscular activity distributions, radial depth positions, circumferential locations, anatomical classifications, and localization confidence parameters. The resulting tissue characterization information is transmitted to the deployment authorization processor following completion of each sensing cycle.

The deployment authorization processor receives tissue characterization information generated during multiple successive sensing cycles and stores the information within a memory structure for comparative evaluation. The processor then analyzes variations occurring among the tissue characterization results obtained over time. Such variations may include changes in radial tissue depth measurements, displacement of identified tissue boundaries, alterations in circumferential location, fluctuations in muscular activity distribution, variations in impedance characteristics, changes in tissue-interface continuity, differences in anatomical classification outcomes, and modifications in confidence values generated by the tissue differentiation processor.

To quantify the consistency of the identified tissue region, the deployment authorization processor determines a localization stability value. The localization stability value represents a measure of how consistently the same tissue structure is identified throughout the plurality of sensing cycles. In certain implementations, the localization stability value may be calculated using statistical variation analysis, temporal correlation algorithms, spatial consistency measurements, confidence aggregation procedures, pattern persistence evaluations, probabilistic localization models, machine-learning classifiers, or combinations thereof. The processor may evaluate the magnitude of positional deviations, the degree of overlap among identified tissue regions, the consistency of anatomical classifications, and the persistence of tissue profile characteristics across successive sensing cycles.

For example, if the tissue differentiation processor repeatedly identifies a circumferential smooth-muscle boundary at substantially the same radial depth and circumferential location during consecutive sensing cycles, the deployment authorization processor may determine that the localization stability value is high. Conversely, if the identified tissue boundary shifts significantly between sensing cycles, exhibits inconsistent anatomical classification results, or demonstrates fluctuating muscular activity patterns, the processor may determine that the localization stability value is low. Such fluctuations may indicate that the identified tissue region has not yet been localized with sufficient certainty for therapeutic deployment.

The deployment authorization processor compares the determined localization stability value against a predetermined acceptable range established for the procedure. The predetermined range may define maximum allowable positional variation, minimum required tissue overlap, acceptable confidence fluctuation limits, permissible anatomical classification variability, or other criteria indicative of stable tissue localization. The predetermined range may be established through calibration procedures, clinical validation studies, patient-specific treatment protocols, or stored operating parameters associated with the device.

While the localization stability value varies beyond the predetermined range, the deployment authorization processor maintains the penetration actuation unit in a deployment-restricted condition. Such restriction may be implemented through retention of a mechanical locking structure, suppression of deployment control signals, prevention of release of a spring-biased actuation mechanism, interruption of electrical power supplied to an actuator, or activation of a software-based deployment interlock. Consequently, even if an operator attempts to initiate therapeutic deployment, advancement of the penetration structure remains prohibited until stable tissue localization has been demonstrated.

The processor continues receiving and evaluating tissue characterization information during additional sensing cycles while the deployment-restricted condition remains active. As new sensing information becomes available, the localization stability value is continuously recalculated and updated. The processor may maintain a rolling analysis window containing a predefined number of recent sensing cycles and evaluate whether the identified tissue characteristics remain substantially unchanged throughout the observation period. Such continuous monitoring enables the system to distinguish persistent anatomical features from transient localization events caused by temporary physiological disturbances.

In certain implementations, the deployment authorization processor may require that the localization stability value remain within the predetermined acceptable range throughout a plurality of consecutive sensing cycles before deployment authorization is granted. This requirement ensures that stability is not demonstrated only momentarily but persists over time. For example, the processor may require successful verification during three, five, ten, or another predetermined number of consecutive sensing cycles. If a significant deviation occurs during any one of the required cycles, the verification sequence may be reset or extended until the required stability criterion is satisfied.

In an exemplary clinical scenario, a first sensing cycle may identify an internal anal sphincter boundary located approximately four millimeters from the distal insertion portion. Subsequent sensing cycles may repeatedly identify the same tissue boundary with minimal variation in radial position, circumferential location, tissue thickness, and electrophysiological activity profile. The deployment authorization processor may determine that the localization stability value remains within the predetermined acceptable range throughout the required sequence of sensing cycles. Upon completion of the verification process, the processor generates a deployment authorization signal permitting operation of the penetration actuation unit.

In another example, the tissue differentiation processor may initially identify a candidate sphincter region; however, subsequent sensing cycles may reveal substantial variation in the location of the identified tissue due to probe displacement, involuntary muscular contraction, patient movement, or sensing noise. As a result, the localization stability value may exceed the permissible range, causing the deployment authorization processor to maintain deployment restriction. Additional sensing cycles are then performed until the identified tissue characteristics stabilize and satisfy the required verification criteria.

By requiring temporal consistency of tissue localization over multiple sensing cycles, the deployment authorization processor introduces a dynamic verification mechanism that complements structural, electrophysiological, impedance-based, and circumferential tissue characterization procedures. The resulting verification architecture improves reliability of tissue identification, reduces susceptibility to transient sensing errors, accommodates physiological motion and anatomical variability, enhances procedural repeatability, and ensures that therapeutic deployment is authorized only after the target internal anal sphincter region has demonstrated sustained localization stability over time.

In an embodiment, the penetration actuation unit is configured to maintain the retractable penetration structure in a retracted position while the deployment authorization processor continues receipt of tissue characterization information from the tissue differentiation processor, initiate advancement of the retractable penetration structure only after receipt of a deployment authorization signal from the deployment authorization processor, and advance the retractable penetration structure along a predetermined deployment path extending through the elongated probe structure toward the identified internal anal sphincter region.

During insertion and positioning of the elongated probe structure within the anal canal, the retractable penetration structure is maintained in a fully retracted position within an internal deployment channel extending through the elongated probe structure. The retractable penetration structure may comprise a hollow injection needle, micro-cannula, therapeutic delivery conduit, fluid delivery shaft, or other penetrating member configured to introduce a therapeutic agent into the identified tissue region. While in the retracted position, the penetration structure remains completely enclosed within the elongated probe structure, thereby preventing unintended contact with surrounding tissue during insertion, repositioning, localization, or sensing operations. The retracted configuration additionally protects the penetration structure from contamination and minimizes the possibility of tissue injury prior to confirmation of the target location.

The penetration actuation unit may include a mechanical retention assembly configured to hold the retractable penetration structure in the retracted position throughout the sensing process. The retention assembly may comprise a locking latch, retention pin, engagement catch, magnetic holding mechanism, electromechanical locking device, spring restraint assembly, or combinations thereof. The locking arrangement prevents accidental advancement of the penetration structure despite external forces, handling movements, or unintentional activation attempts. In certain implementations, the retention assembly remains engaged until a deployment authorization signal is received from the deployment authorization processor.

While the retractable penetration structure remains retracted, the deployment authorization processor continuously receives tissue characterization information generated by the tissue differentiation processor. Such tissue characterization information may be derived from acoustic sensing information, electrophysiological sensing information, impedance sensing information, circumferential tissue mapping data, localization stability measurements, tissue continuity evaluations, and anatomical profile matching procedures. The deployment authorization processor analyzes the received information to determine whether predetermined localization requirements have been satisfied. During this verification phase, multiple sensing cycles may be performed while the penetration structure remains immobilized within the elongated probe structure.

The deployment authorization processor may evaluate various parameters before authorizing deployment, including confirmation of a circumferential smooth-muscle boundary, stability of tissue localization over multiple sensing cycles, correlation between structural and electrophysiological characteristics, continuity of muscular tissue layers, confidence values generated by tissue classification algorithms, and consistency of target identification across different sensing modalities. Until all required verification criteria have been satisfied, the deployment authorization processor maintains a deployment-restricted condition and withholds authorization for penetration structure advancement.

Once the deployment authorization processor determines that the identified tissue region corresponds to the internal anal sphincter with a sufficient degree of confidence, the processor generates a deployment authorization signal. The deployment authorization signal may comprise an electrical control signal, digital command, wireless communication instruction, electromechanical activation command, or equivalent authorization output transmitted to the penetration actuation unit. Receipt of the deployment authorization signal causes the penetration actuation unit to disengage the retention assembly and initiate advancement of the retractable penetration structure.

The initiation of advancement may be accomplished through various actuation mechanisms including spring-driven displacement assemblies, motor-driven translation systems, pneumatic actuators, hydraulic displacement units, electromagnetic drive mechanisms, linear actuators, shape-memory alloy actuators, or combinations thereof. In one implementation, a spring-biased displacement structure stores mechanical energy while the penetration structure remains locked in the retracted position. Upon receipt of the deployment authorization signal, a release mechanism disengages the lock and allows the stored mechanical energy to propel the penetration structure in a controlled manner toward the target tissue region. In another implementation, an electrically powered linear actuator may receive the authorization signal and advance the penetration structure through a precisely controlled displacement sequence.

The retractable penetration structure advances along a predetermined deployment path extending through the elongated probe structure. The deployment path may be defined by a dedicated guide channel, tubular passage, linear guide assembly, reinforced deployment lumen, or precision-machined translation pathway extending from the proximal region of the device to a deployment opening formed in the distal insertion portion. The deployment path constrains movement of the penetration structure to a predefined trajectory and prevents lateral deviation, rotational displacement, bending, or off-axis movement during advancement. Such guidance ensures that the penetration structure travels toward the tissue location identified during the localization process.

The predetermined deployment path may be aligned with the sensing axis used by the tissue sensing unit such that the penetration structure is directed toward the same tissue region that was previously characterized and verified. Consequently, the location identified through acoustic sensing, electrophysiological sensing, impedance analysis, and tissue mapping procedures corresponds substantially to the location reached by the advancing penetration structure. This coordinated alignment between sensing and deployment systems improves targeting accuracy and reduces the likelihood of deviation from the intended therapeutic site.

In certain implementations, the deployment path may further incorporate guide bushings, stabilizing sleeves, anti-rotation elements, low-friction bearing surfaces, alignment rails, or translation supports that maintain accurate positioning throughout advancement. The penetration structure may additionally be advanced at a controlled velocity determined by the actuation mechanism, thereby reducing tissue trauma and ensuring smooth entry into the identified tissue layer. Depth regulation structures positioned along the deployment path may further limit advancement to a predefined penetration distance corresponding to the anatomical dimensions of the identified internal anal sphincter region.

In an exemplary procedure, the tissue differentiation processor may repeatedly identify a smooth-muscle tissue layer exhibiting anatomical and electrophysiological characteristics corresponding to the internal anal sphincter. The deployment authorization processor may subsequently confirm localization stability and circumferential verification requirements over multiple sensing cycles. During this period, the retractable penetration structure remains fully retracted within the elongated probe structure. After the verification requirements are satisfied, the deployment authorization processor transmits a deployment authorization signal to the penetration actuation unit. The actuation unit releases the retention assembly and advances the penetration structure through the predefined deployment path toward the verified tissue region. Once the penetration structure reaches the target location, a therapeutic agent may be delivered directly into the identified internal anal sphincter tissue.

The coordinated interaction between the deployment authorization processor, tissue differentiation processor, and penetration actuation unit establishes a controlled deployment architecture in which therapeutic delivery is preceded by tissue verification, localization validation, and deployment authorization. Such an arrangement improves targeting precision, reduces the possibility of premature or unintended tissue penetration, enhances procedural consistency across different operators, accommodates anatomical variability among patients, and supports accurate placement of therapeutic agents within the intended internal anal sphincter region while minimizing unnecessary tissue disruption.

In an embodiment, the tissue differentiation processor is configured to generate tissue characterization information by continuously updating acoustic response information obtained from the circumferential acoustic sensing arrangement and electrical activity information obtained from the electrophysiological sensing arrangement during movement of the elongated probe structure within the anal canal, determine changes in tissue-interface location and muscular activity distribution occurring during the movement of the elongated probe structure, and identify the internal anal sphincter region from concurrent occurrence of a predetermined tissue-interface pattern and a predetermined muscular activity distribution pattern.

In an embodiment, the tissue differentiation processor is configured to perform dynamic tissue localization by continuously updating tissue characterization information while the elongated probe structure moves within the anal canal. Unlike systems that rely upon measurements obtained only after the probe reaches a stationary position, the present arrangement continuously acquires and processes sensing information during advancement, withdrawal, repositioning, or controlled manipulation of the probe. Such continuous analysis enables real-time assessment of changing anatomical conditions surrounding the distal insertion portion and allows the system to identify the internal anal sphincter region through observation of evolving structural and physiological tissue characteristics encountered during movement through the anal canal.

During operation, the circumferential acoustic sensing arrangement continuously acquires acoustic response information from tissue structures adjacent to the distal insertion portion. The acoustic response information may include tissue-interface positions, radial tissue depths, tissue layer thickness values, acoustic reflection intensities, boundary continuity measurements, tissue morphology information, and circumferential anatomical profiles. As the elongated probe structure moves relative to surrounding tissue, the acoustic sensing arrangement repeatedly generates updated measurements corresponding to newly encountered tissue regions. The signal acquisition processor continuously processes the reflected acoustic signals and updates the acoustic response dataset in real time, thereby producing a dynamic representation of tissue structures encountered along the insertion path.

Simultaneously, the electrophysiological sensing arrangement continuously acquires electrical activity information from tissue surrounding the distal insertion portion. The acquired electrical activity information may include spontaneous muscular activation signals, resting bioelectrical potentials, muscular conduction characteristics, activation frequency distributions, signal amplitudes, waveform characteristics, temporal activation patterns, and circumferential muscular activity distributions. As the probe traverses different anatomical regions, the electrophysiological sensing arrangement detects corresponding changes in tissue activity and continuously updates the electrical activity dataset. Consequently, both structural and functional tissue information are refreshed throughout probe movement rather than being limited to isolated sensing events.

The tissue differentiation processor receives the continuously updated acoustic response information and electrical activity information and maintains a dynamically evolving tissue model representing the anatomical environment surrounding the probe. The processor may utilize real-time signal processing algorithms, temporal tracking procedures, tissue boundary extraction methods, pattern recognition routines, adaptive filtering operations, and anatomical mapping techniques to continuously interpret incoming sensing information. Each newly acquired sensing dataset is compared with previously acquired datasets to determine how tissue characteristics change as the probe progresses through different regions of the anal canal.

As the elongated probe structure moves, the tissue differentiation processor determines changes in tissue-interface location identified from the acoustic response information. Such changes may include variations in radial tissue depth, appearance of new tissue layers, disappearance of previously identified boundaries, alterations in tissue thickness, shifts in circumferential tissue geometry, and modifications in tissue continuity patterns. The processor may track the progression of anatomical structures relative to the distal insertion portion and construct a sequential representation of tissue layers encountered during movement. This enables identification of transitions between adjacent anatomical regions and provides contextual information regarding the spatial arrangement of surrounding tissues.

Concurrently, the tissue differentiation processor evaluates changes in muscular activity distribution derived from the electrophysiological sensing arrangement. The processor monitors how muscular activity characteristics vary as different tissue structures are encountered. Such changes may include increases or decreases in electrical signal amplitude, emergence of sustained smooth-muscle activation patterns, alterations in circumferential activity distribution, changes in conduction behavior, variations in activation frequency, and transitions between involuntary and voluntary muscular activity profiles. By continuously tracking these parameters, the processor develops a dynamic representation of physiological tissue behavior along the sensing path.

The processor subsequently correlates the evolving structural information with the evolving functional information to identify characteristic tissue signatures associated with the internal anal sphincter. Rather than relying upon a single parameter, the processor searches for the concurrent occurrence of a predetermined tissue-interface pattern and a predetermined muscular activity distribution pattern. The predetermined tissue-interface pattern may correspond to an anatomical arrangement known to be associated with the internal anal sphincter, such as the appearance of a circumferential muscular boundary at a particular radial depth beneath a mucosal layer, the presence of a continuous annular tissue structure, a characteristic tissue thickness profile, or a specific sequence of tissue transitions encountered during insertion.

Similarly, the predetermined muscular activity distribution pattern may correspond to physiological behavior characteristic of smooth-muscle sphincter tissue. Such a pattern may include circumferentially distributed involuntary muscular activity, relatively uniform activation characteristics around the circumference, sustained baseline electrical activity, characteristic conduction properties, and activity distributions distinguishable from those associated with skeletal muscle tissue of the external sphincter. The processor continuously compares the acquired muscular activity information against these predefined physiological patterns while simultaneously evaluating the structural tissue configuration identified acoustically.

Identification of the internal anal sphincter region occurs when the processor determines that the predetermined tissue-interface pattern and the predetermined muscular activity distribution pattern occur concurrently within the same anatomical region. In other words, the processor confirms not only that the expected anatomical structure is present but also that the structure exhibits the physiological behavior expected of internal sphincter tissue. This dual-verification process reduces the likelihood that a tissue layer will be incorrectly classified solely on the basis of structural appearance or electrical activity characteristics alone.

In one exemplary implementation, the probe may initially encounter mucosal tissue characterized by superficial acoustic boundaries and relatively limited muscular electrical activity. As advancement continues, the acoustic sensing arrangement may identify the emergence of a continuous circumferential tissue layer located beneath the mucosal interface. Simultaneously, the electrophysiological sensing arrangement may detect the onset of sustained smooth-muscle electrical activity distributed around the circumference of the probe. The processor recognizes the concurrent appearance of the predetermined tissue-interface pattern and the predetermined muscular activity distribution pattern and identifies the corresponding region as the internal anal sphincter.

In an embodiment, the signal acquisition processor is configured to obtain acoustic response information from the circumferential acoustic sensing arrangement during insertion of the distal insertion portion and to generate a plurality of tissue-depth measurements corresponding to different circumferential sensing locations surrounding the elongated probe structure, and wherein the tissue differentiation processor is configured to compare the plurality of tissue-depth measurements acquired from the different circumferential sensing locations, identify a circumferentially extending tissue layer from continuity of the tissue-depth measurements, determine a radial position of the circumferentially extending tissue layer relative to the distal insertion portion, and generate the tissue characterization information according to the determined radial position of the circumferentially extending tissue layer.

During insertion, the signal acquisition processor sequentially activates individual transducer elements according to a predetermined scanning sequence. Each activated transducer element generates an ultrasonic pulse that propagates radially outward into surrounding tissue. As the acoustic energy encounters tissue interfaces characterized by differing acoustic impedances, portions of the transmitted energy are reflected back toward the transducer element. The received echoes are converted into electrical signals and subjected to signal conditioning operations including amplification, filtering, gain compensation, analog-to-digital conversion, noise reduction, and temporal alignment. The processor subsequently determines the time interval between pulse transmission and echo reception and converts the measured time values into tissue-depth measurements using known acoustic propagation characteristics of biological tissue.

The generated tissue-depth measurements represent distances between the circumferential acoustic sensing arrangement and various tissue interfaces located around the distal insertion portion. Such tissue interfaces may correspond to boundaries between mucosal tissue and submucosal tissue, interfaces between connective tissue layers and muscular structures, transitions between smooth muscle and skeletal muscle regions, or other anatomical boundaries encountered during insertion. Because measurements are acquired from numerous circumferential sensing locations, the processor generates a spatially distributed dataset that describes the radial arrangement of tissue structures surrounding the elongated probe structure.

As the distal insertion portion advances through the anal canal, the signal acquisition processor continuously updates the tissue-depth measurements and constructs a circumferential depth profile representative of surrounding anatomy. Each circumferential sensing location contributes a localized depth measurement corresponding to a particular angular position around the probe. The processor associates each depth value with its corresponding circumferential coordinate and organizes the measurements into a structured dataset suitable for subsequent analysis by the tissue differentiation processor. In certain implementations, the processor may perform multiple scanning cycles during insertion to improve measurement reliability and generate averaged depth values that reduce susceptibility to signal noise and transient acoustic artifacts.

The tissue differentiation processor receives the plurality of tissue-depth measurements and performs comparative analysis across the different circumferential sensing locations. Rather than evaluating individual depth measurements independently, the processor analyzes spatial relationships among neighboring measurements to identify recurring anatomical patterns. More specifically, the processor determines whether tissue interfaces detected at multiple circumferential sensing locations exhibit continuity around the circumference of the distal insertion portion. Such continuity analysis is particularly valuable because many anatomical structures within the anal canal, including the internal anal sphincter, extend circumferentially around the canal and therefore produce consistent depth signatures when viewed from multiple angular positions.

The comparison process may involve examination of radial depth consistency, circumferential alignment of tissue interfaces, continuity of detected boundaries, geometric uniformity, tissue layer thickness distributions, and angular persistence of identified structures. The processor may apply interpolation algorithms, circumferential averaging techniques, boundary extraction procedures, curve-fitting operations, spatial continuity assessments, and pattern recognition routines to determine whether a detected tissue interface extends substantially around the circumference of the probe. Tissue interfaces exhibiting continuity across multiple circumferential sensing locations are distinguished from isolated reflections, localized tissue irregularities, anatomical artifacts, or transient measurement anomalies.

When the processor identifies a series of tissue-depth measurements that collectively define a substantially continuous circumferential structure, the processor classifies the detected structure as a circumferentially extending tissue layer. Such a layer may correspond to a muscular ring, circumferential connective tissue structure, or another annular anatomical component surrounding the anal canal. The determination may be based upon the observation that neighboring circumferential sensing locations exhibit tissue interfaces at substantially similar radial distances and that the identified interface remains continuous over a significant portion of the circumference. In certain implementations, the processor may calculate circumferential continuity values, radial variance measurements, geometric coherence indicators, or boundary completeness parameters to quantify the degree of continuity exhibited by the identified layer.

Following identification of the circumferentially extending tissue layer, the tissue differentiation processor determines the radial position of the layer relative to the distal insertion portion. The radial position may be calculated by averaging the tissue-depth measurements associated with the identified layer, determining a circumferential centroid of the layer, identifying minimum and maximum radial extents, or applying geometric reconstruction techniques to model the spatial location of the layer around the probe. The resulting radial position provides information regarding the depth at which the tissue layer is located relative to the sensing elements and therefore relative to the outer surface of the elongated probe structure.

The processor may further compare the determined radial position with stored anatomical reference data corresponding to known tissue structures within the anal canal. Such reference data may include expected radial positions of mucosal layers, submucosal regions, internal anal sphincter tissue, external anal sphincter tissue, connective tissue structures, and other anatomical landmarks. By evaluating the radial position together with continuity characteristics and circumferential geometry, the processor can distinguish among different tissue layers that may otherwise exhibit similar acoustic properties. For example, a circumferentially continuous tissue layer located within a predefined depth range and exhibiting a characteristic annular geometry may correspond to the internal anal sphincter region.

In an exemplary implementation, acoustic sensing may generate thirty-two circumferential tissue-depth measurements during insertion of the distal insertion portion. Analysis of the measurements may reveal a tissue interface located approximately four millimeters from the sensing surface and extending through substantially all circumferential sensing locations. The processor may determine that the interface forms a continuous annular layer exhibiting minimal radial variation around the circumference. Based upon the radial position, continuity characteristics, and correspondence with stored anatomical profiles, the processor may identify the detected layer as the internal anal sphincter. Tissue characterization information is subsequently generated to indicate the location, depth, circumferential extent, and anatomical classification of the identified tissue layer.

In another example, multiple tissue interfaces may be detected at different radial depths. A superficial interface may correspond to mucosal tissue, while a deeper interface may correspond to a circumferential muscular layer. Through comparison of tissue-depth measurements obtained from multiple circumferential sensing locations, the processor may determine that only the deeper interface exhibits the continuity and radial positioning expected of a sphincter structure. Consequently, tissue characterization information is generated based upon the deeper circumferentially extending tissue layer while the superficial interface is excluded from target identification.

By utilizing circumferential acoustic measurements to identify continuous tissue layers and determine their radial positions relative to the distal insertion portion, the tissue differentiation processor generates tissue characterization information that reflects both the geometric organization and anatomical location of surrounding tissue structures. This approach improves recognition of annular muscular anatomy, reduces susceptibility to localized measurement errors, accommodates patient-specific anatomical variability, enhances reliability of tissue layer identification, and provides an accurate structural basis for subsequent localization verification and therapeutic deployment procedures directed toward the internal anal sphincter region.

In an embodiment, the tissue differentiation processor is configured to receive first electrical activity information generated by a first group of the annular conductive sensing elements and second electrical activity information generated by a second group of the annular conductive sensing elements positioned at a different longitudinal location along the distal insertion portion, compare the first electrical activity information and the second electrical activity information to determine variation of muscular activity along a longitudinal sensing path extending adjacent the distal insertion portion, and identify tissue characteristics corresponding to the internal anal sphincter region from the determined variation of muscular activity along the longitudinal sensing path.

In an embodiment, the tissue differentiation processor is configured to perform longitudinal tissue characterization by analyzing electrical activity information acquired from multiple sensing locations positioned at different longitudinal positions along the distal insertion portion. Unlike sensing approaches that evaluate tissue characteristics only at a single circumferential level, the present arrangement enables assessment of how muscular activity changes along the longitudinal axis of the anal canal. Such longitudinal analysis provides additional anatomical information regarding the extent, orientation, and distribution of muscular structures and facilitates more accurate differentiation between the internal anal sphincter and adjacent tissue regions. Because the internal anal sphincter typically extends through a defined longitudinal segment of the anal canal and exhibits characteristic smooth-muscle activity patterns along its length, evaluation of electrical activity variations across multiple longitudinal sensing locations provides a reliable basis for tissue identification.

The electrophysiological sensing arrangement comprises multiple annular conductive sensing elements distributed along the distal insertion portion in a plurality of longitudinally separated sensing groups. A first group of annular conductive sensing elements may be positioned at a first longitudinal location along the distal insertion portion, while a second group of annular conductive sensing elements may be positioned at a second longitudinal location spaced apart from the first location. Additional sensing groups may optionally be provided at further longitudinal positions to increase sensing resolution and expand the anatomical region available for evaluation. The annular conductive sensing elements may be configured as conductive rings, segmented electrode arrays, circumferential sensing bands, microelectrode assemblies, or equivalent conductive sensing structures capable of detecting localized bioelectrical activity generated by adjacent tissue.

During operation, the first group of annular conductive sensing elements acquires first electrical activity information from tissue structures adjacent to the first longitudinal location. Simultaneously or sequentially, the second group of annular conductive sensing elements acquires second electrical activity information from tissue structures adjacent to the second longitudinal location. The acquired electrical activity information may include resting electrical potentials, spontaneous muscular activation signals, signal amplitudes, activation frequencies, conduction characteristics, waveform morphology, phase relationships, synchronization patterns, and temporal muscular response characteristics. Because the sensing groups are positioned at different longitudinal locations, the acquired datasets provide information regarding muscular activity occurring at distinct positions along the longitudinal extent of the anal canal.

The tissue differentiation processor receives the first electrical activity information and the second electrical activity information and organizes the acquired data according to the corresponding longitudinal sensing locations. The processor may perform signal conditioning operations including noise filtering, baseline correction, artifact suppression, amplitude normalization, frequency-domain analysis, temporal averaging, and signal synchronization to facilitate accurate comparison of the datasets. Following preprocessing, the processor evaluates differences and similarities between the electrical activity information obtained from the different longitudinal sensing locations.

The comparison process is directed toward determining variation of muscular activity along a longitudinal sensing path extending adjacent to the distal insertion portion. The longitudinal sensing path represents a virtual sensing axis extending between the first and second sensing groups and corresponding to the anatomical region located between the associated sensing locations. By comparing electrical activity characteristics acquired from different positions along this sensing path, the processor can determine how muscular behavior changes along the length of the tissue being evaluated.

The determined variation may include changes in signal amplitude, differences in activation frequency, alterations in waveform morphology, variations in conduction characteristics, shifts in signal intensity, changes in synchronization behavior, and modifications in temporal activation patterns. For example, a smooth-muscle structure such as the internal anal sphincter may exhibit relatively uniform involuntary electrical activity across a substantial longitudinal segment. Conversely, transitions into adjacent tissue structures may be associated with measurable changes in electrical activity characteristics. The processor therefore analyzes the progression of electrical activity along the longitudinal sensing path to identify locations where tissue properties change and to determine whether the observed activity profile corresponds to known characteristics of internal sphincter tissue.

In certain implementations, the processor generates a longitudinal muscular activity profile representing electrical behavior along the sensing path. The profile may illustrate how muscular activity evolves between the first sensing location and the second sensing location and may identify regions exhibiting consistent activity, increasing activity, decreasing activity, or abrupt activity transitions. The processor may further calculate longitudinal activity gradients, activity continuity values, spatial variation coefficients, activation persistence measurements, and conduction uniformity parameters to quantify the observed muscular behavior.

The tissue differentiation processor may subsequently compare the determined variation of muscular activity with stored anatomical and physiological reference profiles associated with different tissue structures. Such reference profiles may define expected longitudinal activity characteristics of mucosal tissue, connective tissue, internal anal sphincter tissue, external anal sphincter tissue, fibrotic tissue, and other anatomical regions. The processor evaluates the degree of correspondence between the measured activity variation and the reference profiles to identify the tissue structure most consistent with the observed electrical behavior.

In one exemplary implementation, the first sensing group may detect sustained low-frequency involuntary muscular activity at a first longitudinal position while the second sensing group detects substantially similar activity at a second longitudinal position. The processor may determine that the activity remains continuous and relatively uniform along the longitudinal sensing path. Because such a profile corresponds to expected behavior of a smooth-muscle sphincter structure extending longitudinally through the anal canal, the processor may identify the associated tissue region as the internal anal sphincter. The resulting tissue characterization information may include the longitudinal extent of the identified tissue, the location of tissue boundaries, activity continuity measurements, and confidence values associated with the identification.

In another example, the first sensing group may detect activity characteristics associated with smooth muscle tissue, whereas the second sensing group may detect substantially different activity patterns indicative of skeletal muscle tissue or non-muscular tissue. The processor may identify the transition occurring between the two sensing locations and determine that the longitudinal sensing path crosses a tissue boundary. Such information may be used to identify the proximal or distal extent of the internal anal sphincter and to refine localization of the target therapeutic region.

In an embodiment, the deployment authorization processor is configured to receive tissue characterization information generated during advancement of the elongated probe structure and additional tissue characterization information generated after cessation of advancement of the elongated probe structure, compare the tissue characterization information and the additional tissue characterization information to determine persistence of identified tissue characteristics, maintain restriction of the penetration actuation unit when the identified tissue characteristics differ between the tissue characterization information and the additional tissue characterization information, and permit operation of the penetration actuation unit when the identified tissue characteristics remain substantially consistent between the tissue characterization information and the additional tissue characterization information.

In an embodiment, the deployment authorization processor is configured to verify the persistence and repeatability of identified tissue characteristics before permitting activation of the penetration actuation unit. The processor operates on the principle that a correctly identified internal anal sphincter region should remain detectable not only while the elongated probe structure is advancing through the anal canal but also after movement has ceased and the probe has reached a stationary condition. Tissue characteristics that disappear, shift significantly, or exhibit inconsistent behavior following cessation of probe movement may indicate transient sensing artifacts, temporary tissue deformation, motion-induced measurement errors, or inaccurate localization. Accordingly, the deployment authorization processor performs a comparative verification procedure using tissue characterization information acquired under both dynamic and stationary sensing conditions.

During advancement of the elongated probe structure, the tissue sensing unit continuously acquires acoustic response information, electrophysiological activity information, impedance information, or combinations thereof from surrounding tissue structures. The tissue differentiation processor analyzes the acquired sensing data and generates tissue characterization information representing tissue boundaries, tissue depth values, muscular activity distributions, circumferential tissue continuity, anatomical classifications, localization coordinates, and confidence metrics associated with an identified tissue region. This information reflects tissue conditions observed while the probe is moving relative to the surrounding anatomy and therefore captures dynamic interactions occurring during insertion and positioning.

The deployment authorization processor receives the tissue characterization information generated during advancement and stores the information within an associated memory structure. The stored information may include radial tissue positions, circumferential coordinates, longitudinal locations, tissue-interface geometries, muscular activity profiles, tissue classification outputs, confidence values, and anatomical correspondence measurements. These parameters collectively define the characteristics of the tissue region identified during probe advancement and establish a reference dataset for subsequent verification procedures.

After advancement of the elongated probe structure ceases, the distal insertion portion remains substantially stationary within the anal canal. In this stationary condition, additional sensing cycles are performed to obtain updated information regarding the tissue structures surrounding the probe. The tissue sensing unit continues acquiring acoustic and electrophysiological information while motion-induced influences are substantially reduced. The tissue differentiation processor processes the newly acquired sensing information and generates additional tissue characterization information corresponding to the tissue region identified after cessation of movement. Because the probe is no longer advancing, the additional tissue characterization information reflects tissue characteristics under a more stable sensing environment and therefore provides an independent confirmation of the previously identified target region.

The deployment authorization processor receives the additional tissue characterization information and performs a comparative analysis between the tissue characterization information generated during advancement and the additional tissue characterization information generated after movement has ceased. The comparison may involve evaluation of radial depth measurements, circumferential tissue distributions, longitudinal tissue positions, tissue-interface continuity, muscular activity patterns, impedance profiles, tissue classification outcomes, and confidence values. The processor determines whether the same anatomical structure has been identified under both sensing conditions and whether the detected tissue characteristics persist despite the transition from dynamic movement to stationary observation.

To determine persistence of the identified tissue characteristics, the processor may calculate positional correspondence values, geometric overlap metrics, anatomical similarity scores, activity consistency measurements, confidence correlation values, or composite persistence indicators. In certain implementations, the processor evaluates whether the identified tissue boundaries remain substantially unchanged, whether muscular activity distributions continue to exhibit the expected physiological characteristics, and whether the tissue classification remains consistent across both sensing conditions. Persistence is therefore assessed not merely on the basis of location but also on the basis of structural and functional similarity.

For example, during advancement of the probe, the tissue differentiation processor may identify a circumferential smooth-muscle layer located at a particular radial depth and exhibiting characteristic electrophysiological activity associated with the internal anal sphincter. After the probe reaches a stationary position, subsequent sensing cycles may reveal the same tissue boundary at substantially the same radial location together with a similar smooth-muscle activity profile. The deployment authorization processor may determine that the identified tissue characteristics persist across both sensing conditions and therefore conclude that the localization result is reliable.

Conversely, if tissue characterization information generated during advancement identifies a candidate internal sphincter region but the additional tissue characterization information generated after cessation of movement identifies a substantially different tissue boundary, different muscular activity distribution, or different anatomical classification, the processor may determine that the identified tissue characteristics are not persistent. Such discrepancies may indicate that the original identification was influenced by probe motion, temporary tissue displacement, transient physiological activity, signal noise, or localized sensing artifacts. In such circumstances, the processor maintains restriction of the penetration actuation unit and prevents deployment of the penetration structure until additional sensing confirms a stable and repeatable localization result.

The restriction of the penetration actuation unit may be implemented through maintenance of a mechanical locking mechanism, continued engagement of a deployment interlock, suppression of deployment control signals, prevention of actuator energization, or retention of a spring-biased actuation assembly in a locked state. As long as the identified tissue characteristics differ between the tissue characterization information generated during advancement and the additional tissue characterization information generated after cessation of movement, the deployment authorization processor prevents activation of the therapeutic deployment mechanism. This restriction serves as an additional verification layer that supplements tissue differentiation, circumferential verification, localization stability assessment, and multimodal tissue characterization procedures.

When the processor determines that the identified tissue characteristics remain substantially consistent between the tissue characterization information acquired during advancement and the additional tissue characterization information acquired after movement has ceased, the persistence requirement is considered satisfied. Substantial consistency may be determined when positional deviations remain within predefined limits, tissue classifications remain unchanged, muscular activity distributions exhibit corresponding patterns, and anatomical boundaries demonstrate acceptable spatial agreement. Upon satisfaction of these criteria, the deployment authorization processor generates a deployment authorization signal permitting operation of the penetration actuation unit.

In an exemplary clinical implementation, the probe may be advanced until the tissue differentiation processor identifies a candidate internal anal sphincter region based upon acoustic and electrophysiological information. The deployment authorization processor stores the corresponding tissue characterization information and subsequently instructs the operator to temporarily hold the probe stationary. Additional sensing cycles are then performed. If the same tissue region continues to be identified with substantially identical structural and functional characteristics, the processor determines that the tissue characteristics persist under both dynamic and stationary conditions and authorizes deployment. If the identified characteristics change significantly following cessation of movement, deployment authorization is withheld until a stable target region is verified.

In an embodiment, the depth regulation unit is configured to establish a predetermined maximum advancement distance for the retractable penetration structure relative to a deployment position of the therapeutic delivery unit, and wherein the penetration actuation unit is configured to displace the retractable penetration structure from a retracted position toward the deployment opening while the depth regulation unit continuously limits displacement of the retractable penetration structure, the depth regulation unit terminating advancement of the retractable penetration structure upon attainment of the predetermined maximum advancement distance irrespective of continued operation of the penetration actuation unit.

The depth regulation unit may comprise one or more mechanical depth-control components positioned within the deployment pathway of the retractable penetration structure. Such components may include adjustable stop members, penetration-limiting collars, threaded depth regulators, calibrated mechanical barriers, displacement-limiting sleeves, depth-control cams, travel-limiting rails, telescopic stop assemblies, or combinations thereof. These structures are configured to define a maximum permissible travel distance for the retractable penetration structure measured from its fully retracted position or from a predefined deployment reference position within the therapeutic delivery unit.

Prior to deployment, the depth regulation unit establishes the predetermined maximum advancement distance based upon anatomical measurements, tissue characterization information, physician-selected treatment settings, factory calibration parameters, stored procedural protocols, or combinations thereof. In certain implementations, the predetermined maximum advancement distance may correspond to the estimated depth of the internal anal sphincter identified by the tissue differentiation processor. For example, if tissue characterization procedures determine that the target muscular layer is positioned approximately four millimeters beneath the tissue surface adjacent to the deployment opening, the depth regulation unit may be adjusted to permit advancement slightly sufficient to place the distal end of the retractable penetration structure within the identified muscular region while preventing further penetration beyond the desired therapeutic zone.

The penetration actuation unit is configured to displace the retractable penetration structure from a retracted position toward the deployment opening after receipt of a valid deployment authorization signal. The actuation unit may utilize spring-driven mechanisms, motor-driven actuators, pneumatic systems, hydraulic displacement assemblies, electromagnetic actuators, shape-memory alloy actuators, or equivalent deployment mechanisms capable of generating translational movement. During advancement, the retractable penetration structure travels through a guided deployment pathway extending toward the deployment opening located in the distal insertion portion.

As advancement occurs, the depth regulation unit continuously limits displacement of the retractable penetration structure. Unlike systems in which penetration depth is controlled solely by timing, actuator power, or operator judgment, the present arrangement provides continuous mechanical regulation throughout the deployment process. The depth regulation unit remains engaged during the entire advancement sequence and actively monitors or constrains movement of the retractable penetration structure. In certain implementations, physical interaction between a moving component of the penetration actuation unit and a stationary depth-limiting structure determines the allowable travel distance. In other implementations, displacement measurements may be continuously evaluated against a predefined threshold while a corresponding mechanical stop remains available as a fail-safe limitation mechanism.

The depth regulation unit may further incorporate calibration features enabling precise control of advancement distance. Such features may include graduated depth indicators, indexed adjustment positions, detent-based selection mechanisms, threaded adjustment scales, numerical reference markings, locking collars, or digital displacement indicators. These calibration features enable selection of penetration depths corresponding to different patient anatomies, tissue thicknesses, treatment protocols, or therapeutic objectives. Once a depth setting has been selected, the depth regulation unit maintains the selected limitation throughout the procedure and prevents unauthorized modification during deployment.

As the retractable penetration structure approaches the predetermined maximum advancement distance, the depth regulation unit progressively constrains further movement. Upon attainment of the predetermined maximum advancement distance, the depth regulation unit physically terminates advancement of the retractable penetration structure regardless of whether the penetration actuation unit continues to generate force. The termination of advancement may occur through direct engagement of a stop surface, contact between a moving carriage and a limiting barrier, engagement of a threaded stop member, activation of a locking mechanism, or mechanical interference between corresponding displacement-control components. Consequently, movement of the retractable penetration structure ceases even if residual spring energy, motor torque, pneumatic pressure, or other actuation force remains present within the penetration actuation unit.

In one implementation, the penetration actuation unit may comprise a spring-biased displacement mechanism capable of generating sufficient force to advance the retractable penetration structure through tissue. During deployment, the spring-driven mechanism continues exerting force throughout the advancement sequence. However, once the retractable penetration structure reaches the predetermined maximum advancement distance established by the depth regulation unit, a depth-limiting stop member engages the moving carriage assembly and prevents further displacement. The remaining stored energy within the spring mechanism is therefore unable to produce additional advancement beyond the predefined limit.

In another example, a motor-driven actuation mechanism may continue operating momentarily after the retractable penetration structure reaches the predetermined advancement distance. Despite continued operation of the motor, engagement of the depth regulation unit prevents further penetration movement. The mechanical limitation therefore functions independently of the actuator control system and provides an additional layer of protection against excessive penetration resulting from software errors, control signal anomalies, actuator malfunction, or operator error.

In certain embodiments, the depth regulation unit may be configured to accommodate different anatomical dimensions identified during tissue localization. For example, a patient exhibiting a relatively thin internal anal sphincter may require a shorter maximum advancement distance than a patient exhibiting a thicker muscular layer. The depth regulation unit may therefore be adjusted according to tissue-depth information generated by the tissue differentiation processor. This individualized depth control enables the penetration structure to reach the therapeutic target while minimizing unnecessary penetration into adjacent structures such as the external sphincter, connective tissue layers, vascular regions, or surrounding anatomical components.

In an exemplary procedure, the tissue sensing unit identifies the radial position of the internal anal sphincter and communicates the corresponding tissue-depth information to the deployment control system. The depth regulation unit is adjusted to establish a maximum advancement distance corresponding to the identified anatomical depth. Following receipt of deployment authorization, the penetration actuation unit advances the retractable penetration structure from its retracted position toward the deployment opening and subsequently into the identified tissue region. Throughout advancement, the depth regulation unit continuously constrains displacement. Once the predetermined advancement distance is reached, the depth regulation unit terminates movement of the penetration structure even though the actuation mechanism remains capable of generating additional force. Therapeutic delivery may then be performed with the penetration structure positioned substantially within the intended tissue layer.

By providing continuous displacement limitation and positive termination of penetration movement at a predefined distance, the depth regulation unit establishes a highly controlled deployment environment. This arrangement improves consistency of therapeutic placement, minimizes the risk of excessive tissue penetration, reduces dependence upon operator technique, accommodates patient-specific anatomical variations, enhances procedural safety, and ensures that therapeutic delivery occurs within the intended internal anal sphincter region despite continued operation or residual force generation by the penetration actuation unit.

In an embodiment, the tissue differentiation processor is configured to generate a plurality of tissue characterization results from corresponding sets of tissue response information acquired by the tissue sensing unit during a plurality of sensing cycles, determine a confidence value according to a degree of agreement among the plurality of tissue characterization results, transmit the confidence value to the deployment authorization processor together with the tissue characterization information, and wherein the deployment authorization processor is configured to compare the confidence value with a deployment criterion and permit extension of the retractable penetration structure only when the confidence value satisfies the deployment criterion and the tissue characterization information identifies the internal anal sphincter region.

During operation, the tissue sensing unit acquires corresponding sets of tissue response information during a plurality of sensing cycles performed while the elongated probe structure is positioned within the anal canal. Each sensing cycle may include acquisition of acoustic response information from the circumferential acoustic sensing arrangement, electrophysiological activity information from the electrophysiological sensing arrangement, impedance response information from the impedance sensing arrangement, or combinations thereof. The acquired information may represent tissue-interface locations, tissue depth measurements, circumferential tissue continuity characteristics, muscular activity distributions, electrical resistance profiles, tissue morphology parameters, and other physiological or structural attributes associated with surrounding anatomical structures.

For each sensing cycle, the tissue differentiation processor independently analyzes the acquired tissue response information and generates a corresponding tissue characterization result. Each tissue characterization result may include identification of a candidate tissue region, anatomical classification information, radial depth coordinates, circumferential location data, longitudinal position information, muscular activity characteristics, tissue boundary definitions, tissue transition zone locations, and associated classification confidence parameters. Since the tissue characterization process is repeated across multiple sensing cycles, a plurality of tissue characterization results is generated, each representing an independent assessment of the surrounding tissue environment.

The processor subsequently evaluates the degree of agreement among the plurality of tissue characterization results. The agreement determination may involve comparison of anatomical classifications, spatial coordinates, tissue-interface locations, tissue depth measurements, muscular activity patterns, circumferential continuity values, impedance characteristics, and other tissue-identification parameters generated during the various sensing cycles. The processor may determine whether the same tissue region is repeatedly identified and whether the identified characteristics remain substantially consistent throughout the sensing sequence.

In certain implementations, the degree of agreement may be calculated through statistical analysis techniques including variance determination, standard deviation calculations, probability distribution analysis, weighted consensus evaluation, Bayesian inference models, confidence aggregation algorithms, machine-learning classification consistency analysis, or pattern correlation procedures. The processor may evaluate the extent to which the plurality of tissue characterization results converge toward a common tissue identification outcome. Tissue characterization results exhibiting minimal variation and high spatial correspondence indicate strong agreement, whereas results exhibiting substantial variation suggest uncertainty in tissue localization.

Based upon the determined degree of agreement, the tissue differentiation processor generates a confidence value representing the reliability of the identified tissue region. The confidence value may comprise a numerical score, probability estimate, confidence percentage, weighted classification metric, normalized agreement index, or other quantitative representation of localization certainty. Higher confidence values are generated when multiple sensing cycles consistently identify substantially the same anatomical structure and associated tissue characteristics. Lower confidence values are generated when the tissue characterization results exhibit inconsistencies, conflicting classifications, or significant positional variations.

The confidence value may incorporate contributions from multiple evaluation criteria. For example, the processor may consider the consistency of tissue depth measurements, stability of circumferential tissue boundaries, persistence of muscular activity distributions, repeatability of tissue-interface identification, agreement among impedance measurements, and correspondence with stored anatomical reference profiles. The resulting confidence value therefore reflects a comprehensive assessment of both structural and functional tissue characteristics rather than a single measurement parameter.

Following generation of the confidence value, the tissue differentiation processor transmits the confidence value together with the corresponding tissue characterization information to the deployment authorization processor. The transmitted information may include the identified tissue classification, localization coordinates, tissue boundary definitions, muscular activity characteristics, anatomical profile data, and the associated confidence value. By providing both the tissue characterization result and the confidence assessment, the deployment authorization processor is able to evaluate not only which tissue has been identified but also how reliably that identification has been established.

The deployment authorization processor receives the confidence value and compares it with a predefined deployment criterion. The deployment criterion may comprise a minimum confidence threshold, required probability level, minimum agreement score, predefined consensus value, statistical reliability threshold, or other acceptance criterion established through calibration procedures, clinical validation studies, or procedural safety requirements. The deployment criterion represents the minimum degree of localization certainty required before therapeutic deployment may be permitted.

When the confidence value remains below the deployment criterion, the deployment authorization processor maintains restriction of the retractable penetration structure. Such restriction may be implemented through maintenance of a mechanical locking assembly, prevention of actuator activation, retention of a spring-biased deployment mechanism in a locked condition, interruption of deployment control signals, or activation of an electronic deployment interlock. Additional sensing cycles may be performed to obtain further tissue response information and improve localization confidence. As new sensing results become available, the tissue differentiation processor recalculates the confidence value and transmits updated information to the deployment authorization processor.

Only when two conditions are simultaneously satisfied does the deployment authorization processor permit extension of the retractable penetration structure. First, the confidence value must satisfy the deployment criterion, thereby indicating that the identified tissue location has been established with sufficient certainty. Second, the tissue characterization information must identify the internal anal sphincter region as the intended therapeutic target. Satisfaction of one condition alone is insufficient. For example, a high confidence value associated with identification of a non-target tissue structure will not result in deployment authorization. Similarly, identification of a candidate internal anal sphincter region will not result in authorization if the associated confidence value remains below the required threshold.

In an exemplary implementation, the tissue sensing unit may perform ten successive sensing cycles while the elongated probe structure remains positioned adjacent a candidate target region. The tissue differentiation processor may determine that nine of the ten sensing cycles identify substantially the same circumferential smooth-muscle structure at nearly identical radial and longitudinal positions. The resulting degree of agreement produces a confidence value exceeding the predetermined deployment criterion. Because the tissue characterization information also identifies the structure as the internal anal sphincter region, the deployment authorization processor generates a deployment authorization signal and permits extension of the retractable penetration structure.

In another example, successive sensing cycles may produce inconsistent tissue characterization results due to probe movement, transient muscular contractions, localized inflammation, or sensing noise. Although some sensing cycles may indicate the presence of internal sphincter tissue, the overall degree of agreement among the results remains insufficient to satisfy the deployment criterion. Consequently, the deployment authorization processor continues to restrict deployment and requires acquisition of additional sensing information until localization confidence improves.

By integrating repeated tissue characterization, agreement-based confidence determination, and confidence-dependent deployment authorization, the system establishes a hierarchical verification architecture that significantly strengthens tissue localization reliability. This arrangement reduces the likelihood of deployment based upon isolated sensing events, enhances discrimination between target and non-target tissues, accommodates patient-specific anatomical variability, improves procedural repeatability, and ensures that therapeutic deployment is performed only after both reliable identification and high-confidence verification of the internal anal sphincter region have been achieved.

For specification support, it is advisable to include a dedicated hardware implementation description covering the physical realization of the various functional units. A suitable paragraph is provided below:

The system may comprise an elongated probe structure formed from a biocompatible polymeric, metallic, composite, or elastomeric material configured for insertion into an anal canal and housing a plurality of integrated sensing, processing, deployment, and therapeutic delivery components. The circumferential acoustic sensing arrangement may comprise a plurality of ultrasound transducer elements mounted within or upon the distal insertion portion and electrically connected to signal transmission conductors extending through the elongated probe structure. The ultrasound transducer elements may be implemented as piezoelectric, capacitive, microelectromechanical, or equivalent acoustic transduction devices configured to generate and receive acoustic energy from surrounding tissue structures. The electrophysiological sensing arrangement may comprise annular conductive sensing elements, segmented electrode arrays, conductive rings, microelectrodes, conductive traces, or conductive contact regions fabricated from electrically conductive biocompatible materials and positioned along circumferential and longitudinal locations of the distal insertion portion for acquisition of localized electrical activity information. The impedance sensing arrangement may comprise dedicated excitation electrodes, measurement electrodes, switching circuitry, analog front-end circuits, current generation circuitry, voltage sensing circuitry, impedance measurement modules, and associated conductive pathways configured to introduce controlled excitation signals into surrounding tissue and measure resultant electrical response characteristics.

The signal acquisition processor may be implemented using one or more microprocessors, microcontrollers, digital signal processors, field-programmable logic devices, application-specific integrated circuits, programmable logic controllers, embedded computing modules, or combinations thereof. Associated hardware may further include analog-to-digital converters, digital-to-analog converters, multiplexers, amplifiers, signal conditioning circuits, filters, clock generation circuits, memory devices, data buffers, and communication interfaces configured to receive, process, store, and transmit sensing information. The tissue differentiation processor may comprise dedicated computational hardware including processing cores, memory modules, mathematical acceleration circuits, pattern-recognition engines, artificial intelligence accelerators, programmable logic resources, or equivalent electronic processing architectures configured to execute tissue classification, anatomical mapping, tissue correlation, and localization determination operations. In certain implementations, the signal acquisition processor and tissue differentiation processor may be incorporated within a common electronic control assembly or may be implemented as separate hardware modules interconnected through wired or printed circuit interconnections.

The deployment authorization processor may comprise a dedicated control processor, embedded controller, programmable logic circuit, state-machine controller, safety interlock controller, or equivalent electronic authorization module configured to receive tissue characterization information and generate deployment control instructions. The deployment authorization processor may further include non-volatile memory, volatile memory, comparator circuits, verification logic circuitry, watchdog circuits, fault-monitoring circuitry, and safety control interfaces configured to evaluate localization confidence and control operation of the penetration actuation unit. The tissue sensing unit may therefore comprise an integrated assembly of sensing elements, acquisition electronics, signal routing circuits, power regulation circuitry, shielding structures, and processing hardware positioned within the elongated probe structure and operatively coupled to the associated processors.

The therapeutic delivery unit may comprise a therapeutic reservoir chamber formed as a cartridge, chamber, vial receptacle, collapsible reservoir, piston-actuated reservoir, or fluid storage cavity configured to contain a therapeutic composition. The therapeutic reservoir chamber may be fluidically connected to a hollow penetration structure through one or more fluid conduits, microfluidic channels, delivery lumens, valves, check valves, flow-regulating components, seals, gaskets, and fluid transfer passages. The hollow penetration structure may comprise a hollow needle, microcannula, tubular penetration member, fluid delivery shaft, or equivalent penetrating component fabricated from metallic, polymeric, ceramic, or composite materials. The hollow penetration structure may be positioned within a guided translation pathway formed by a deployment lumen, guide channel, tubular sleeve, bearing-supported track, rail assembly, or equivalent guidance structure extending longitudinally through the elongated probe structure toward a deployment opening located in the distal insertion portion.

The penetration actuation unit may comprise one or more mechanical or electromechanical displacement mechanisms including compression springs, torsional springs, extension springs, pneumatic actuators, hydraulic actuators, linear motors, rotary motors coupled to translation assemblies, electromagnetic actuators, shape-memory alloy actuators, lead-screw assemblies, rack-and-pinion mechanisms, cam-driven actuators, or combinations thereof configured to generate controlled displacement of the retractable penetration structure. The penetration actuation unit may further include guided carriage assemblies, translation members, bearing structures, guide rails, alignment bushings, anti-rotation elements, locking members, release mechanisms, trigger assemblies, retention structures, displacement sensors, position encoders, and motion-control components configured to regulate advancement and retraction of the penetration structure. The selectively releasable locking structure may comprise latches, catches, locking pins, magnetic retention assemblies, electromechanical locking devices, solenoid-operated retainers, mechanical engagement structures, or equivalent restraint mechanisms configured to maintain the penetration structure in a retracted condition until deployment authorization is received.

The depth regulation unit may comprise adjustable penetration limiting structures including threaded stop members, adjustable collars, depth-control sleeves, travel-limiting barriers, calibrated stop assemblies, telescoping regulators, detent-based positioning mechanisms, indexed positioning elements, ratcheting assemblies, rotational adjustment structures, displacement limiters, and mechanical depth-control interfaces configured to establish and maintain a predetermined maximum advancement distance for the penetration structure. The movable penetration barrier may include threaded engagement surfaces, calibrated markings, locking rings, rotational adjustment knobs, tactile indexing mechanisms, and position-retention components configured to permit user-selectable penetration settings corresponding to anatomical requirements. In certain implementations, the depth regulation unit may further comprise displacement sensors, encoder assemblies, limit switches, position monitoring circuits, or other hardware configured to monitor penetration depth during deployment.

The system may further comprise power supply circuitry including rechargeable batteries, primary batteries, inductive power receivers, wired power interfaces, voltage regulation circuits, power management modules, charging circuitry, isolation circuits, and protection circuitry configured to provide operating power to the sensing, processing, and deployment subsystems. Additional hardware may include communication modules, wired interfaces, wireless transceivers, display assemblies, operator input interfaces, visual indicators, audio indicators, memory storage devices, printed circuit boards, electromagnetic shielding structures, protective housings, sealing assemblies, sterilizable enclosures, and environmental protection structures configured to support reliable operation of the system during tissue localization and therapeutic delivery procedures. This hardware architecture enables coordinated acquisition of structural and physiological tissue information, verification of tissue localization, controlled deployment of the penetration structure, and accurate delivery of therapeutic agents to the identified internal anal sphincter region.

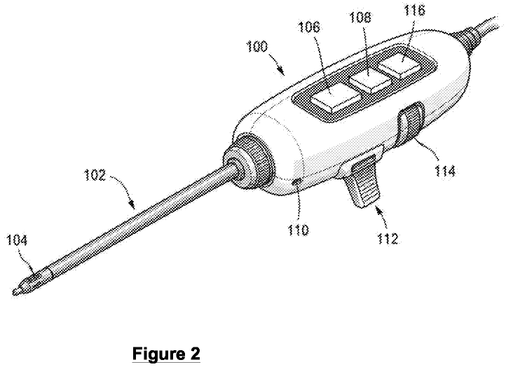

FIG. 2 illustrates a perspective view of a tissue-targeted therapeutic delivery device (100) configured to perform localized tissue sensing, target differentiation, controlled penetration, and therapeutic delivery within a selected anatomical region. The device (100) includes an elongated probe structure (102) extending distally from a handheld body and configured for insertion into tissue. The elongated probe structure (102) may possess a substantially cylindrical geometry and may include rigid, semi-rigid, or flexible construction materials suitable for guided advancement within biological structures. A tissue sensing unit (104) is positioned at a distal region of the elongated probe structure (102). The tissue sensing unit (104) may include one or more sensing components configured to detect tissue characteristics, physiological signatures, acoustic properties, electrical properties, impedance characteristics, or combinations thereof. The tissue sensing unit (104) continuously acquires local tissue information during insertion and transmits acquired signals for further processing.

The device (100) further includes a signal acquisition processor (106) positioned within the body portion of the device. The signal acquisition processor (106) receives signals generated by the tissue sensing unit (104) and performs signal acquisition, conditioning, amplification, filtering, digitization, and preprocessing operations. Acquired data are communicated to a tissue differentiation processor (108), which is configured to analyze signal characteristics and distinguish anatomical structures or tissue boundaries based upon predetermined computational criteria. The tissue differentiation processor (108) may implement tissue classification operations and identify a target treatment region based upon characteristic signal patterns.

A therapeutic delivery unit (110) is integrated within the device body and is configured to store, guide, or deliver a therapeutic composition. The therapeutic delivery unit (110) may include fluid channels, reservoirs, injection pathways, or deployment structures for administration of therapeutic agents. A penetration actuation unit (112) is coupled to the therapeutic delivery unit (110) and controls mechanical advancement of a penetration member or delivery structure toward target tissue. The penetration actuation unit (112) may include trigger-actuated, motor-driven, spring-assisted, electromechanical, or manually operated deployment mechanisms.

The device further includes a depth regulation unit (114) configured to control insertion positioning and penetration depth during advancement of the elongated probe structure (102). The depth regulation unit (114) may include adjustable mechanical stops, collars, movable sleeves, sensing feedback structures, or depth calibration arrangements configured to prevent excessive penetration. A deployment authorization processor (116) evaluates information generated by the tissue differentiation processor (108) and determines whether predefined deployment conditions have been satisfied before activation of therapeutic delivery. The deployment authorization processor (116) thereby restricts deployment until the desired target tissue location has been identified.

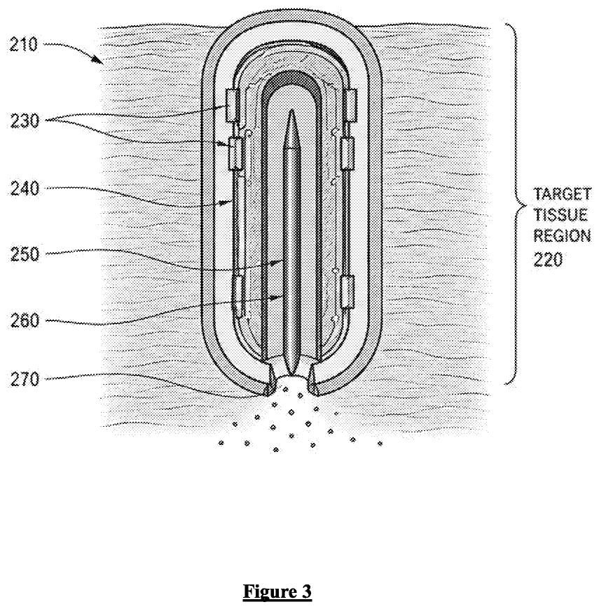

FIG. 3 illustrates a cross-sectional view of a distal probe portion (210) positioned within a target tissue region (220). The distal probe portion (210) forms a terminal section of the elongated probe structure and includes internal sensing and therapeutic delivery structures configured for localized tissue interrogation and treatment administration. The distal probe portion (210) may include a multilayer structure defining internal channels and embedded sensing elements.

A plurality of tissue sensing elements (230) is positioned around a peripheral sensing region within the distal probe portion (210). The tissue sensing elements (230) are configured to acquire tissue-specific information from surrounding tissue structures and may include acoustic sensors, impedance sensors, electrical sensing elements, pressure sensors, optical sensors, or combinations thereof. The tissue sensing elements (230) communicate acquired information through a signal transmission pathway (240). The signal transmission pathway (240) extends longitudinally through the distal probe portion (210) and provides electrical communication between sensing elements and processing circuitry located within the body of the device.

A therapeutic delivery channel (250) extends through a central region of the distal probe portion (210). The therapeutic delivery channel (250) provides a guided pathway for movement of a deployable penetration member (260). The deployable penetration member (260) may include a needle, injector, penetrating element, therapeutic delivery structure, or equivalent tissue access member configured for selective advancement into surrounding tissue. Advancement of the deployable penetration member (260) occurs in response to activation of the penetration actuation unit after target confirmation has been achieved.

A therapeutic release opening (270) is provided near a distal end of the probe portion and is configured to permit controlled administration of a therapeutic composition into the target tissue region (220). The therapeutic release opening (270) may include one or more apertures, side ports, micro-openings, or discharge channels configured to release therapeutic substances in a controlled manner. The illustrated arrangement enables simultaneous tissue sensing, target differentiation, penetration control, and localized therapeutic administration while minimizing unintended deployment into adjacent anatomical structures.

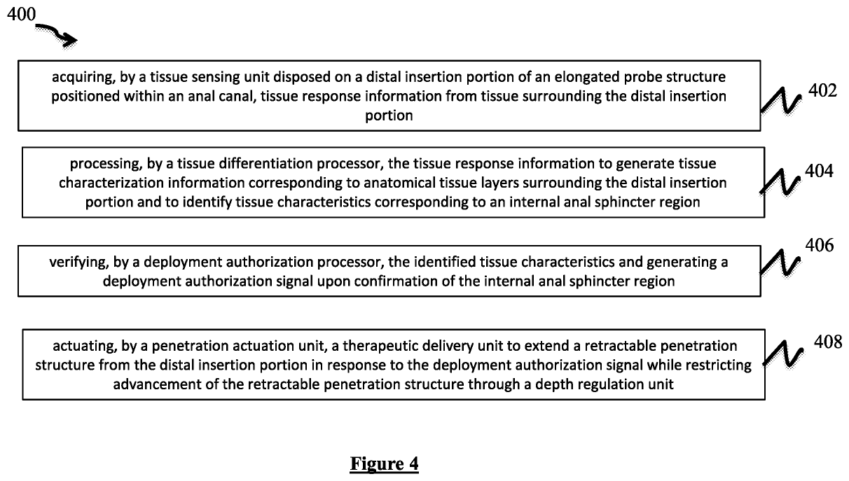

FIG. 4 illustrates a flow chart of a method for targeted delivery of a therapeutic agent into an internal anal sphincter region, the method comprises the steps of:
At step 402, the method 400 includes acquiring, by a tissue sensing unit disposed on a distal insertion portion of an elongated probe structure positioned within an anal canal, tissue response information from tissue surrounding the distal insertion portion;
At step 404, the method 400 includes processing, by a tissue differentiation processor, the tissue response information to generate tissue characterization information corresponding to anatomical tissue layers surrounding the distal insertion portion and to identify tissue characteristics corresponding to an internal anal sphincter region;
At step 406, the method 400 includes verifying, by a deployment authorization processor, the identified tissue characteristics and generating a deployment authorization signal upon confirmation of the internal anal sphincter region;
At step 408, the method 400 includes actuating, by a penetration actuation unit, a therapeutic delivery unit to extend a retractable penetration structure from the distal insertion portion in response to the deployment authorization signal while restricting advancement of the retractable penetration structure through a depth regulation unit; and
At step 410, the method 400 includes delivering the therapeutic agent through the retractable penetration structure into the confirmed internal anal sphincter region.

In an embodiment, the internal anal sphincter localization and injection system is implemented as a physically integrated medical apparatus composed of structural, electromechanical, sensing, and control hardware assemblies arranged within a probe-based therapeutic device architecture. The elongated probe structure is implemented as a hardware insertion assembly manufactured from a biocompatible semi-rigid polymeric body, medical-grade composite material, or metallic tubular structure having defined mechanical stiffness characteristics for maintaining structural integrity during insertion and positioning operations. The proximal handling portion is implemented as a rigid housing structure containing internal compartments configured for accommodation of electrical conductors, fluid channels, mechanical transmission members, and electronic circuitry, while the distal insertion portion comprises a physically formed extension section incorporating sensing structures, deployment openings, and tissue interaction surfaces. The tissue sensing unit is implemented as a hardware sensing arrangement physically mounted on or embedded within the distal insertion portion and includes actual sensing elements such as ultrasound transducers, conductive electrode structures, impedance measurement contacts, pressure sensing elements, or combinations thereof, arranged as fixed physical components around the probe body. The ultrasound transducers comprise piezoelectric crystal structures configured to convert electrical excitation into acoustic energy and reflected acoustic signals into electrical signals. Electrophysiological sensing components comprise conductive metallic sensing pads or annular electrodes mounted beneath insulating layers and electrically connected through conductor pathways. Impedance sensing structures comprise conductive tissue-contact terminals configured to generate electrical excitation and receive measurable tissue resistance characteristics.

The signal acquisition processor is implemented as dedicated electronic hardware comprising signal-conditioning circuitry, analog interface circuitry, analog-to-digital conversion circuitry, timing control components, electronic filtering structures, and printed circuit assemblies configured to physically receive electrical signals from the tissue sensing unit. Such hardware circuitry acquires and conditions incoming sensing information prior to transmission for tissue analysis operations. The tissue differentiation processor is implemented as a physically embodied electronic processing assembly comprising a microprocessor circuit, integrated circuit packages, memory structures, hardware signal processing circuitry, and associated electronic interconnections mounted on a circuit substrate. The tissue differentiation processor receives digitized tissue information and performs tissue characterization operations through execution of instruction sets stored within physically embodied memory devices. The deployment authorization processor is similarly implemented as a dedicated hardware processing structure including processor circuitry, memory units, electronic logic circuitry, and communication interfaces configured to generate deployment enabling or blocking signals based upon received tissue identification information.

The therapeutic delivery unit is implemented as a hardware fluid delivery assembly comprising a hollow penetration structure, a therapeutic reservoir chamber, fluid conduits, sealing structures, and mechanically guided support members physically arranged within the elongated probe structure. The hollow penetration structure is implemented as a metallic cannula or tubular penetration member fabricated from corrosion-resistant biocompatible material and configured to physically extend from the distal insertion portion for tissue penetration and therapeutic delivery. The therapeutic reservoir chamber comprises a physically formed storage cavity or cartridge housing configured to contain injectable therapeutic compositions and provide fluid communication with the hollow penetration structure through interconnected conduits. The penetration actuation unit is implemented as a hardware displacement mechanism comprising mechanical structures including spring elements, guided carriage members, linear displacement members, rotational transmission components, actuator housings, or electromechanical movement assemblies configured to physically generate controlled movement of the hollow penetration structure. Such actuation hardware converts stored mechanical energy or supplied electrical energy into translational movement required for penetration deployment operations.

The depth regulation unit is implemented as a physically adjustable penetration limiting assembly including threaded stop members, movable collars, indexed positioning structures, telescopic limiting members, rotational adjustment components, or mechanical barriers configured to establish a predefined maximum penetration distance. These hardware structures physically obstruct or terminate movement of the penetration structure beyond selected displacement limits and thereby regulate tissue penetration depth. The orientation sensing unit is implemented as a hardware positional sensing arrangement including angular position sensors, rotational displacement elements, orientation conductors, indexed rotational markers, or electromechanical sensing components configured to physically detect rotational orientation of the probe body relative to surrounding anatomical structures. The tissue contact pressure sensing unit is implemented using pressure-responsive hardware components including strain sensing structures, flexible membrane sensing elements, piezoresistive components, or force-responsive conductive structures physically positioned on external probe surfaces to detect tissue interaction characteristics.

The mechanical interlock control unit is implemented as a hardware locking arrangement comprising physical engagement structures, movable locking members, latching components, retention structures, and release mechanisms configured to selectively obstruct movement of the penetration actuation unit. The physical locking member comprises a movable mechanical element configured to transition between an engaged state and a disengaged state in response to electronic control signals generated by processor circuitry. The circumferential tissue mapping unit is implemented through hardware arrangements including segmented sensing regions, annular sensor rings, electrical interconnection pathways, and multiple sensing structures physically distributed around the probe circumference. The multiple axially separated sensing rings are implemented as physically spaced annular assemblies mounted at longitudinal intervals along the distal insertion portion and configured to generate tissue information at distinct spatial locations. Internal longitudinal passageways are implemented as physically formed channels, conduits, lumens, and separated compartments extending through the probe body to accommodate wiring structures, fluid transport pathways, sensing conductors, and mechanical displacement structures. Accordingly, each constituent element of the system is implemented as a physically realizable hardware structure possessing defined material characteristics, geometric configurations, and mechanical or electronic functionality to permit construction and operation of the integrated localization and therapeutic delivery apparatus.

In an embodiment, operation of the internal anal sphincter localization and injection system is governed by a tissue localization and deployment control procedure executed through coordinated interaction among sensing hardware, signal acquisition circuitry, processing circuitry, actuation assemblies, and safety regulation structures. The procedure begins upon insertion of the elongated probe structure into the anal canal and activation of sensing circuitry located within the distal insertion portion. Following activation, the signal acquisition processor initiates synchronized acquisition of tissue response information from the tissue sensing unit. The sensing unit continuously obtains anatomical information from surrounding tissue structures through multiple sensing modalities including acoustic sensing structures, electrophysiological sensing structures, impedance sensing structures, and pressure sensing structures. Since tissue boundaries are often difficult to identify using a single sensing technique, acquisition of multiple forms of tissue information permits generation of a more robust and anatomically accurate characterization process.

In one embodiment, the signal acquisition processor begins a sensing cycle by transmitting timing synchronization signals to each sensing structure positioned around the circumferential surface of the distal insertion portion. Acoustic sensing structures positioned around the probe body sequentially emit controlled acoustic pulses toward adjacent tissue regions. Following acoustic transmission, reflected signals generated from tissue interfaces are received by the acoustic sensing structures and converted into electrical response signals. Simultaneously, electrophysiological sensing structures establish temporary contact with surrounding tissue surfaces and obtain electrical activity signatures generated by nearby muscular structures. Impedance sensing structures generate low-amplitude excitation signals and measure corresponding tissue resistance characteristics across surrounding tissue regions. Pressure sensing structures additionally obtain tissue contact information representing interaction forces occurring between the distal insertion portion and surrounding anatomical surfaces.

The signal acquisition processor receives the electrical outputs generated by the sensing structures and performs preprocessing operations before tissue characterization analysis begins. In one embodiment, preprocessing operations include signal amplification, electronic noise suppression, signal normalization, analog filtering, digitization operations, and temporal synchronization processing. Acoustic signals obtained from ultrasound transducers may undergo gain adjustment operations and electronic compensation to improve reflected signal clarity. Electrophysiological sensing information may undergo noise filtering to remove unwanted electrical disturbances associated with tissue movement and environmental interference. Impedance information may undergo compensation processing to eliminate measurement deviations caused by tissue hydration variability and surface contact conditions. Temporal synchronization procedures align sensing information generated from multiple sensing sources to permit comparative evaluation of corresponding tissue regions.

Following signal acquisition and preprocessing operations, the tissue differentiation processor initiates tissue characterization procedures. Initially, the tissue differentiation processor separates incoming sensing information into tissue-specific datasets corresponding to distinct sensing categories. Acoustic response information is processed to determine tissue depth characteristics and identify reflected signal transition regions corresponding to interfaces between adjacent tissue layers. The processor identifies amplitude variations, signal propagation delays, reflection intensity characteristics, and spatial discontinuities associated with anatomical boundaries. Tissue interface locations are subsequently estimated through comparative analysis of detected reflection patterns.

Electrophysiological information obtained from surrounding tissue structures undergoes activity characterization processing. During such processing, the tissue differentiation processor identifies muscular response characteristics associated with smooth muscular tissue and skeletal muscular tissue. Signal amplitude patterns, temporal activation characteristics, spatial signal distributions, and muscular response signatures are evaluated to distinguish tissue types. Since the internal anal sphincter primarily consists of smooth muscular tissue and the external anal sphincter consists of skeletal muscular tissue, distinct electrophysiological characteristics permit tissue differentiation through pattern analysis procedures.

Simultaneously, impedance sensing information undergoes resistance profile generation procedures. The tissue differentiation processor receives electrical response information obtained from tissue contact regions and determines tissue impedance distributions across multiple circumferential sensing locations. Spatial variations in tissue resistance are evaluated to identify transitions between mucosal structures and muscular structures. The processor additionally compensates for tissue hydration effects and contact pressure variations by incorporating tissue interaction information generated by pressure sensing structures. Such compensation reduces measurement deviations and improves tissue characterization consistency.

After independent processing of sensing information, the tissue differentiation processor initiates tissue fusion procedures through operation of a tissue profile comparison unit. During this procedure, tissue information generated from acoustic sensing, electrophysiological sensing, impedance sensing, and pressure sensing structures is combined into a composite tissue characterization dataset. The tissue profile comparison unit assigns relative weighting values to tissue parameters according to confidence values associated with each sensing source. For example, if acoustic signal quality decreases due to tissue movement or reflection distortion, greater weighting may be assigned to electrophysiological or impedance information. Alternatively, if tissue contact conditions become inconsistent, pressure sensing information may reduce influence of affected impedance measurements. Dynamic weighting procedures permit adaptive tissue characterization under changing anatomical conditions.

The tissue profile comparison unit subsequently generates tissue classification information through comparison of composite tissue characteristics with stored tissue reference information located within memory structures associated with the tissue differentiation processor. Tissue reference information includes predetermined tissue boundary characteristics, muscular activity characteristics, tissue thickness characteristics, impedance distributions, and anatomical transition profiles associated with mucosal structures, internal sphincter tissue, and external sphincter tissue. Similarity evaluation procedures determine correspondence between acquired tissue information and stored tissue patterns. The processor continuously updates confidence values associated with tissue identification decisions throughout insertion and positioning operations.

In an embodiment incorporating circumferential tissue mapping structures, the tissue differentiation processor additionally generates spatial tissue distribution information using sensing data acquired from segmented sensing regions positioned around the distal insertion portion. Tissue information obtained from multiple angular positions is organized according to rotational orientation information generated by the orientation sensing unit. The processor combines circumferential sensing information with longitudinal sensing information generated from axially separated sensing rings to construct a volumetric representation of surrounding tissue structures. Tissue boundary contours and muscular regions are thereby reconstructed across multiple spatial dimensions.

Following tissue characterization procedures, the deployment authorization processor receives tissue identification information and determines whether conditions required for therapeutic deployment have been satisfied. The processor evaluates tissue confidence values, positional consistency characteristics, pressure information, and tissue localization stability measurements. In one embodiment, tissue confirmation is established only after repeated acquisition cycles produce tissue identification information exceeding predetermined confidence thresholds over multiple consecutive measurement intervals. This repeated verification process minimizes erroneous deployment arising from temporary sensing disturbances or anatomical movement.

When deployment conditions remain unsatisfied, the deployment authorization processor maintains engagement of a mechanical interlock structure positioned adjacent the penetration actuation unit. The interlock structure physically obstructs displacement of the retractable penetration structure and prevents tissue penetration. Continuous sensing procedures continue while the elongated probe structure undergoes rotational or positional adjustments by a user.

Following confirmation of internal anal sphincter tissue identification, the deployment authorization processor generates a deployment authorization signal transmitted toward the penetration actuation unit. Upon receipt of the authorization signal, the penetration actuation unit initiates controlled displacement of the retractable penetration structure. Mechanical movement may occur through release of stored spring energy, operation of a linear displacement mechanism, or activation of electromechanical movement structures. Movement of the penetration structure occurs along a predetermined translation pathway constrained by guide structures positioned within the elongated probe body.

During penetration advancement, movement of the retractable penetration structure is monitored by the depth regulation unit. Mechanical limiting structures including threaded stop members, indexed barriers, and displacement restriction components establish a predefined maximum penetration distance corresponding to selected anatomical dimensions. Once penetration reaches the predetermined depth limit, further advancement is mechanically prevented. Therapeutic material contained within the therapeutic reservoir chamber is subsequently delivered through the hollow penetration structure into the identified tissue region.

Following completion of therapeutic administration, retraction procedures are initiated. The penetration actuation unit withdraws the hollow penetration structure into a protected internal position within the elongated probe body. Tissue sensing operations may continue during retraction to verify procedural completion and monitor tissue interaction characteristics. Upon completion of therapeutic delivery, the elongated probe structure is withdrawn from the anatomical region, thereby concluding the localization and therapeutic administration sequence. Through coordinated operation of sensing hardware, tissue characterization procedures, adaptive tissue fusion operations, safety confirmation procedures, and controlled deployment structures, the system provides improved localization accuracy, procedural repeatability, and reduced dependence upon operator estimation.

The internal anal sphincter localization and injection device comprises a machine structure having an elongated insertion assembly manufactured from a biocompatible semi-rigid material configured to permit controlled insertion within an anal canal while maintaining sufficient structural stability during localization and injection procedures.

The insertion assembly includes a proximal handle portion comprising a grip structure having an ergonomic contour adapted to permit stable manipulation during therapeutic procedures. Internal passageways extend longitudinally within the handle and probe structures and accommodate electrical conductors, signal pathways, fluid delivery channels, actuator transmission members, and deployment components.

Disposed near the distal end of the insertion assembly is a tissue differentiation architecture comprising multiple tissue sensing structures arranged circumferentially around the probe surface. In one embodiment, miniature ultrasound transducers are distributed around the distal portion and configured to emit acoustic signals toward surrounding tissue regions. Reflected signals generated by tissue interfaces are collected and transformed into tissue depth information.

The ultrasound sensing arrangement operates in conjunction with electrode arrays configured to obtain muscular activity signatures associated with tissue structures. The electrode structures comprise annular conductive members embedded beneath an insulating protective layer and positioned to establish temporary contact with tissue surfaces. Electrical response patterns generated by muscular tissue structures are collected and processed to identify distinct tissue characteristics.

In another embodiment, impedance sensing structures are integrated into the probe exterior. These sensing structures generate electrical field distributions and evaluate tissue resistance profiles. Variations in impedance values corresponding to different tissue compositions permit discrimination among mucosal tissue, smooth muscular tissue associated with the internal anal sphincter, and skeletal muscular tissue associated with external sphincter structures.

The distal localization structure further includes a circumferential tissue mapping ring configured to combine sensing outputs into a composite tissue profile. The mapping ring may comprise multiple independently segmented sensing zones positioned radially around the insertion assembly. Tissue data collected from the zones permits three-dimensional characterization of surrounding anatomical structures.

A therapeutic delivery assembly is integrated within the insertion structure and comprises a retractable penetration member configured to extend through a radial deployment opening. The penetration member may comprise a hollow needle fabricated from corrosion-resistant biocompatible material and fluidically connected to a therapeutic reservoir structure.

The needle assembly is mounted upon a controlled advancement mechanism positioned within the probe body. The advancement mechanism may comprise a spring-biased carriage, threaded translation member, linear actuator arrangement, rotational cam mechanism, or guided slider architecture. Controlled displacement of the mechanism advances the needle through a predetermined travel pathway.

A depth control structure is mechanically coupled to the deployment mechanism. The depth control structure comprises adjustable penetration limiting members configured to define a maximum needle advancement distance. Such penetration control members may include sliding collars, indexed stop members, telescoping penetration barriers, or rotational depth adjustment structures.

In a preferred embodiment, penetration depth is constrained within a range corresponding to anatomical dimensions associated with the internal anal sphincter region, thereby reducing probability of penetration into deeper structures.

The device further includes a control architecture housed within the proximal section. The control architecture includes signal acquisition circuitry, sensor interface components, electrical conditioning structures, processing circuitry, actuator drivers, and deployment enabling structures.

The control architecture continuously receives tissue information from the sensing assembly and evaluates tissue profiles through comparative signal processing operations. Upon identification of tissue characteristics corresponding to internal anal sphincter tissue, a deployment authorization signal activates a mechanical release structure permitting therapeutic administration.

If target tissue identification remains incomplete, an interlock structure maintains deployment restriction through engagement of a mechanical locking member. The locking member physically obstructs advancement of the injection mechanism and prevents unintentional deployment.

In an additional embodiment, the device incorporates rotational indexing structures permitting angular repositioning of the sensing assembly relative to surrounding tissue. Such structures include rotational rings, indexing detents, and orientation markers configured to facilitate controlled alignment with selected anatomical positions.

The therapeutic material delivery system may contain pharmacological agents including muscular relaxation compositions, regenerative formulations, anti-inflammatory compounds, local anesthetic materials, tissue healing agents, or neurotoxic agents configured to reduce sphincter hypertonicity.

During operation, the insertion assembly is introduced into the anal canal and advanced toward a treatment region. Tissue sensing structures continuously acquire surrounding tissue information while the localization architecture generates tissue characterization profiles. Upon positive identification of internal anal sphincter tissue, the control architecture releases the deployment interlock. The therapeutic needle advances through a controlled pathway and delivers therapeutic material into the identified tissue region. Following administration, the needle retracts into the protective housing structure and the device is withdrawn.

The integrated machine structure thereby provides an accurate and repeatable tissue localization and therapeutic delivery mechanism with reduced procedural variability and improved treatment precision.

The present invention generally relates to medical localization and therapeutic delivery devices and, more particularly, relates to a probe-based internal anal sphincter localization and injection system configured for targeted identification of anatomical tissue structures and controlled administration of therapeutic agents within anorectal regions. More specifically, the invention relates to a structurally integrated medical apparatus incorporating tissue sensing hardware, signal acquisition circuitry, tissue differentiation processors, penetration control structures, and therapeutic delivery assemblies configured to identify internal anal sphincter tissue and perform localized therapeutic administration with improved positional accuracy and procedural safety. The invention further relates to minimally invasive tissue localization and guided intervention systems employing multimodal tissue characterization and controlled deployment arrangements for treatment of anorectal conditions including chronic anal fissures and related sphincter-associated disorders.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. An internal anal sphincter localization and injection system for targeted delivery of a therapeutic agent into an internal anal sphincter region, the system comprising:
an elongated probe structure comprising a proximal handling portion and a distal insertion portion configured for placement within an anal canal;
a tissue sensing unit disposed at the distal insertion portion and comprising a plurality of tissue interaction elements positioned circumferentially around an outer surface of the elongated probe structure;
a signal acquisition processor electrically coupled to the tissue sensing unit and configured to receive tissue response information generated by the plurality of tissue interaction elements;
a tissue differentiation processor operatively coupled to the signal acquisition processor and configured to generate tissue characterization information corresponding to anatomical tissue layers surrounding the distal insertion portion;
a therapeutic delivery unit positioned within an internal cavity of the elongated probe structure and comprising a retractable penetration structure configured to selectively extend from a deployment opening formed in the distal insertion portion;
a penetration actuation unit mechanically coupled to the therapeutic delivery unit and configured to produce controlled displacement of the retractable penetration structure;
a depth regulation unit coupled to the penetration actuation unit and configured to restrict advancement distance of the retractable penetration structure; and
a deployment authorization processor operatively coupled to the tissue differentiation processor and the penetration actuation unit, wherein the deployment authorization processor is configured to permit extension of the retractable penetration structure only after identification of tissue characteristics corresponding to an internal anal sphincter region, wherein the tissue sensing unit comprises a circumferential acoustic sensing arrangement including a plurality of high-frequency ultrasound transducer elements distributed at angular intervals around the distal insertion portion, each high-frequency ultrasound transducer element being configured to emit acoustic signals toward surrounding tissue structures and receive reflected signals representative of tissue interface boundaries, and wherein the signal acquisition processor is configured to generate spatial tissue profile information from time-varying acoustic reflections obtained from the plurality of high-frequency ultrasound transducer elements, and wherein the tissue sensing unit further comprises an electrophysiological sensing arrangement including multiple annular conductive sensing elements embedded beneath an insulating surface layer disposed around the elongated probe structure, the conductive sensing elements being configured to establish temporary contact with adjacent tissue and detect electrical activity associated with muscular tissue structures, and wherein the tissue differentiation processor is configured to distinguish smooth muscle tissue characteristics from skeletal muscle tissue characteristics using electrical activity variations acquired through the conductive sensing elements, and wherein the depth regulation unit is configured to establish a predetermined maximum advancement distance for the retractable penetration structure relative to a deployment position of the therapeutic delivery unit, and wherein the penetration actuation unit is configured to displace the retractable penetration structure from a retracted position toward the deployment opening while the depth regulation unit continuously limits displacement of the retractable penetration structure, the depth regulation unit terminating advancement of the retractable penetration structure upon attainment of the predetermined maximum advancement distance irrespective of continued operation of the penetration actuation unit, and wherein the tissue differentiation processor is configured to generate a plurality of tissue characterization results from corresponding sets of tissue response information acquired by the tissue sensing unit during a plurality of sensing cycles, determine a confidence value according to a degree of agreement among the plurality of tissue characterization results, transmit the confidence value to the deployment authorization processor together with the tissue characterization information, and wherein the deployment authorization processor is configured to compare the confidence value with a deployment criterion and permit extension of the retractable penetration structure only when the confidence value satisfies the deployment criterion and the tissue characterization information identifies the internal anal sphincter region.

2. The system of claim 1, wherein the electrophysiological sensing arrangement further comprises circumferentially segmented electrode regions positioned in axially separated rows along the distal insertion portion, each segmented electrode region being individually addressable by the signal acquisition processor to permit sequential acquisition of localized tissue activity information and generation of three-dimensional muscular activity distribution data corresponding to surrounding anatomical structures, and wherein the tissue sensing unit comprises an impedance sensing arrangement including multiple conductive contact regions configured to introduce electrical excitation signals into adjacent tissue structures and measure electrical resistance characteristics associated with tissue composition, and wherein the tissue differentiation processor is configured to determine transitions among mucosal tissue, internal sphincter tissue, and external sphincter tissue based upon measured electrical response characteristics and spatial impedance distribution information.

3. The system of claim 1, wherein the tissue differentiation processor further comprises a tissue profile comparison unit configured to receive acoustic sensing information, electrophysiological sensing information, and impedance sensing information and generate composite tissue characterization information through weighted comparison of multiple tissue parameters including tissue depth characteristics, muscular activity characteristics, and electrical resistance characteristics, and wherein the depth regulation unit further comprises a movable penetration barrier including indexed positioning elements and rotational adjustment structures configured to permit selection of multiple penetration settings corresponding to distinct anatomical dimensions and patient-specific tissue characteristics.

4. The system of claim 1, wherein the therapeutic delivery unit comprises a hollow penetration structure fluidically coupled to a therapeutic reservoir chamber disposed within the proximal handling portion, and wherein the hollow penetration structure is positioned within a guided translation pathway extending longitudinally through the elongated probe structure and terminating at the deployment opening formed in the distal insertion portion, wherein the penetration actuation unit comprises a spring-biased displacement structure, a guided carriage assembly, and a selectively releasable locking structure configured to convert stored mechanical force into controlled translational displacement of the hollow penetration structure, and wherein the guided carriage assembly constrains movement of the hollow penetration structure along a predetermined deployment axis, and wherein the depth regulation unit comprises an adjustable penetration limiting structure including a threaded stop member positioned adjacent the guided carriage assembly and configured to establish a maximum translational displacement distance corresponding to predefined tissue penetration depths associated with anatomical dimensions of an internal anal sphincter region.

5. The system of claim 1, wherein the signal acquisition processor is configured to sequentially activate individual high-frequency ultrasound transducer elements of the circumferential acoustic sensing arrangement disposed within the tissue sensing unit, receive reflected acoustic signals corresponding to a plurality of circumferential tissue locations surrounding the distal insertion portion, generate acoustic response information representative of tissue-interface positions at the plurality of circumferential tissue locations, and transmit the acoustic response information to the tissue differentiation processor, and wherein the tissue differentiation processor is configured to compare tissue-interface positions identified from the plurality of circumferential tissue locations to determine continuity of a muscular tissue boundary surrounding the distal insertion portion and generate the tissue characterization information based upon the determined continuity of the muscular tissue boundary.

6. The system of claim 1, wherein the signal acquisition processor is configured to acquire electrical activity information from the annular conductive sensing elements of the electrophysiological sensing arrangement during a plurality of successive acquisition intervals occurring while the elongated probe structure is positioned within the anal canal, and wherein the tissue differentiation processor is configured to organize the electrical activity information according to circumferential sensing location, determine spatial variation of muscular activity surrounding the distal insertion portion, compare the spatial variation of muscular activity with acoustic response information generated from the circumferential acoustic sensing arrangement, and identify tissue characteristics corresponding to the internal anal sphincter region from a spatial correlation between the muscular activity information and the acoustic response information.

7. The system of claim 1, wherein the tissue differentiation processor is configured to receive acoustic response information generated from the circumferential acoustic sensing arrangement and electrical activity information generated from the electrophysiological sensing arrangement, determine a plurality of candidate tissue transition locations from the acoustic response information, determine a plurality of muscular tissue locations from the electrical activity information, compare the candidate tissue transition locations with the muscular tissue locations, and generate the tissue characterization information only after identification of a spatial relationship between the candidate tissue transition locations and the muscular tissue locations corresponding to an internal anal sphincter tissue profile.

8. The system of claim 1, wherein the deployment authorization processor is configured to receive a first tissue characterization information set generated during a first sensing cycle, receive a second tissue characterization information set generated during a subsequent sensing cycle, compare the first tissue characterization information set and the second tissue characterization information set to determine positional consistency of an identified internal anal sphincter region, maintain restriction of the penetration actuation unit while the positional consistency remains below a predetermined verification condition, and permit operation of the penetration actuation unit after the positional consistency satisfies the predetermined verification condition.

9. The system of claim 1, wherein the deployment authorization processor is configured to receive tissue characterization information corresponding to a plurality of circumferential sensing locations surrounding the distal insertion portion, determine whether tissue characteristics corresponding to the internal anal sphincter region are detected at multiple circumferential sensing locations, generate a circumferential verification result from the detected tissue characteristics, and permit extension of the retractable penetration structure only after the circumferential verification result satisfies a predefined tissue-localization condition.

10. The system of claim 1, wherein the tissue differentiation processor is configured to generate a circumferential tissue map by associating acoustic response information acquired from the circumferential acoustic sensing arrangement with electrical activity information acquired from the electrophysiological sensing arrangement at corresponding circumferential sensing locations, identify a tissue transition zone separating a smooth muscle region from an adjacent tissue region within the circumferential tissue map, and generate the tissue characterization information from the identified tissue transition zone.

11. The system of claim 1, wherein the deployment authorization processor is configured to receive the tissue characterization information from the tissue differentiation processor during a plurality of successive sensing cycles, determine a localization stability value according to variation in tissue characteristics identified during the plurality of successive sensing cycles, maintain the penetration actuation unit in a deployment-restricted condition while the localization stability value varies beyond a predetermined range, and permit operation of the penetration actuation unit after the localization stability value remains within the predetermined range throughout a plurality of consecutive sensing cycles.

12. The system of claim 1, wherein the penetration actuation unit is configured to maintain the retractable penetration structure in a retracted position while the deployment authorization processor continues receipt of tissue characterization information from the tissue differentiation processor, initiate advancement of the retractable penetration structure only after receipt of a deployment authorization signal from the deployment authorization processor, and advance the retractable penetration structure along a predetermined deployment path extending through the elongated probe structure toward the identified internal anal sphincter region.

13. The system of claim 1, wherein the tissue differentiation processor is configured to generate tissue characterization information by continuously updating acoustic response information obtained from the circumferential acoustic sensing arrangement and electrical activity information obtained from the electrophysiological sensing arrangement during movement of the elongated probe structure within the anal canal, determine changes in tissue-interface location and muscular activity distribution occurring during the movement of the elongated probe structure, and identify the internal anal sphincter region from concurrent occurrence of a predetermined tissue-interface pattern and a predetermined muscular activity distribution pattern.

14. The system of claim 1, wherein the signal acquisition processor is configured to obtain acoustic response information from the circumferential acoustic sensing arrangement during insertion of the distal insertion portion and to generate a plurality of tissue-depth measurements corresponding to different circumferential sensing locations surrounding the elongated probe structure, and wherein the tissue differentiation processor is configured to compare the plurality of tissue-depth measurements acquired from the different circumferential sensing locations, identify a circumferentially extending tissue layer from continuity of the tissue-depth measurements, determine a radial position of the circumferentially extending tissue layer relative to the distal insertion portion, and generate the tissue characterization information according to the determined radial position of the circumferentially extending tissue layer.

15. The system of claim 1, wherein the tissue differentiation processor is configured to receive first electrical activity information generated by a first group of the annular conductive sensing elements and second electrical activity information generated by a second group of the annular conductive sensing elements positioned at a different longitudinal location along the distal insertion portion, compare the first electrical activity information and the second electrical activity information to determine variation of muscular activity along a longitudinal sensing path extending adjacent the distal insertion portion, and identify tissue characteristics corresponding to the internal anal sphincter region from the determined variation of muscular activity along the longitudinal sensing path.

16. The system of claim 1, wherein the deployment authorization processor is configured to receive tissue characterization information generated during advancement of the elongated probe structure and additional tissue characterization information generated after cessation of advancement of the elongated probe structure, compare the tissue characterization information and the additional tissue characterization information to determine persistence of identified tissue characteristics, maintain restriction of the penetration actuation unit when the identified tissue characteristics differ between the tissue characterization information and the additional tissue characterization information, and permit operation of the penetration actuation unit when the identified tissue characteristics remain substantially consistent between the tissue characterization information and the additional tissue characterization information.
